(19) United States
(12) Patent Application Publication
(10) Pub. No.: US 2025/0333486 A1
(43) Pub. Date: Oct. 30, 2025

(54) Anti-HSP90 alpha antibody and uses thereof

(71) Applicant: NATIONAL HEALTH RESEARCH INSTITUTES, Miaoli County (TW)

(72) Inventors: Tze-Sing HUANG, Miaoli County (TW); Tsu-An HSU, Miaoli County (TW); Hui-Chen HUNG, Miaoli County (TW); Yi-Yu KE, Miaoli County (TW)

(21) Appl. No.: 18/710,913

(22) PCT Filed: Nov. 22, 2021
(86) PCT No.: PCT/US2021/060295

§ 371 Date: May 16, 2024

(51) Int. Cl. C07K 16/18; A61K 31/7068; A61K 39/00; A61K 39/395; A61P 35/00

(52) U.S. Cl. CPC C07K 16/18; A61K 31/7068; A61K 39/39558; A61P 35/00; A61K 2039/505; C07K 2317/24; C07K 2317/34; C07K 2317/54; C07K 2317/92

(57) ABSTRACT

Disclosed is an isolated antibody comprises: novel complementarity determining regions capable of specifically binding to the HSP90α epitope containing two EDK sites in the amino acid 235 to 244 and amino acid 251 to 260 regions, respectively. Also disclosed are nucleic acid molecules corresponding to the aforesaid antibody, a pharmaceutical composition comprising the aforesaid antibody or the corresponding nucleic acid molecules, and the methods for treating and monitoring a cancer using the aforesaid antibody.

### SEQUENCE LISTING INCORPORATION BY REFERENCE STATEMENT

A sequence listing ASCII text file is incorporated herein by reference. The sequence listing ASCII file is named “HUAN3876_ReplacementSequenceListing.txt,” created May 15, 2024, and revised Nov. 27, 2024. The size of the sequence listing ASCII text file is 9534 bytes.

### FIELD OF THE DISCLOSURE

The present disclosure relates to a novel anti-HSP90α antibody and uses thereof. More specifically, the present relates to a novel isolated anti-HSP90α antibody, a pharmaceutical composition comprising the same and a method for treating a cancer using the same.

### BACKGROUND

Cancer development and progression depend not only on genetic and epigenetic alterations in epithelial cells but also on critical changes in their stromal microenvironment which consists of extracellular matrix (ECM) and stromal cells like fibroblasts and immune cells. See Pandol S, Edderkaoui M, Gukovsky I, Lugea A, Gukovskaya A. Desmoplasia of pancreatic ductal adenocarcinoma. Clin Gastroenterol Hepatol 2009;7(11):S44-7. Desmoplasia is a common characteristic of many malignancies such as pancreatic ductal adenocarcinoma (PDAC) and colorectal carcinoma (CRC) and results from large amounts of ECM as well as great numbers of myofibroblasts which express α-smooth muscle actin (α-SMA) as a defining marker. Such myofibroblasts, also called activated fibroblasts or cancer-associated fibroblasts (CAFs), contribute to tumor growth, immunosuppression, and malignant progression. See Orimo A, Gupta P B, Sgroi D C, Arenzana-Seisdedos F, Delaunay T, Naeem R, et al. Stromal fibroblasts present in invasive human breast carcinomas promote tumor growth and angiogenesis through elevated SDF-1/CXCL12 secretion. Cell 2005;121(3):335-48; Lakins M A, Ghorani E, Munir H, Carla P. Martins CP, Shields J D. Cancer-associated fibroblasts induce antigen-specific deletion of CD8⁺ T cells to protect tumour cells. Nat Commun 2018;9(1):948; Turley S J, Cremasco V, Astarita J L. Immunological hallmarks of stromal cells in the tumour microenvironment. Nat Rev Immunol 2015;15(11):669-82; and Beacham D A, Cukierman E. Stromagenesis: the changing face of fibroblastic microenvironments during tumor progression. Semin Cancer Biol 2005;15(5):329-41. They constitute the majority of tumor stromal cells and can be derived from diverse resources such as tissue-resident fibroblasts, stellate cells, mesenchymal stem/progenitor cells, and infiltrating fibrocytes. See Sugimoto H, Mundel T M, Kieran M W, Kalluri R. Identification of fibroblast heterogeneity in the tumor microenvironment. Cancer Biol Ther 2006;5(12):1640-6. Additionally, CAFs can also arise from the endothelial-to-mesenchymal transition (EndoMT) of endothelial cells. See Zeisberg E M, Potenta S, Xie L, Zeisberg M, Kalluri R. Discovery of endothelial to mesenchymal transition as a source for carcinoma-associated fibroblasts. Cancer Res 2007;67(21):10123-8. In our previous study, EndoMT-derived CAFs (i.e., α-SMA⁺CD31⁺ cells) were detected nearby osteopontin (OPN)-expressing macrophages in CRC tissue specimens. See Fan C S, Chen W S, Chen L L, Chen C C, Hsu Y T, Chua K V, et al. Osteopontin-integrin engagement induces HIF-1α-TCF12-mediated endothelial-mesenchymal transition to exacerbate colorectal cancer. Oncotarget 2018;9(4):4998-5015. OPN induced the EndoMT of endothelial cells and the resultant EndoMT-derived CAFs exhibited a potent tumor-promoting effect by secreting HSP90α to foster CRC cell stemness. See Fan C S, Chen W S, Chen L L, Chen C C, Hsu Y T, Chua K V, et al. Osteopontin-integrin engagement induces HIF-1α-TCF12-mediated endothelial-mesenchymal transition to exacerbate colorectal cancer. Oncotarget 2018;9(4):4998-5015. Recently, we also found that the mix of EndoMT-derived CAFs with PDAC cell grafts significantly recruited myeloid-derived macrophages, prevented immune T cells, and promoted tumor growth. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138. HSP90α secreted by EndoMT-derived CAFs further induced macrophage M2-polarization and more HSP90α secretion through cell-surface receptors CD91 and TLR4 and the downstream MyD88-JAK2/TYK2-STAT-3 pathway. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138.

HSP90α is a well-known cellular chaperone aiding the folding, maturation, and trafficking of many client proteins including cancer-related Bcr-Abl, ErbB2/Neu, Akt, HIF-1α, mutated p53, and Raf-1. See Trepel J B, Mollapour M, Giaccone G, Neckers L. Targeting the dynamic Hsp90 complex in cancer. Nat Rev Cancer 2010;10(8):537-49. It can also be expressed and secreted from the keratinocytes and fibroblasts in wounded tissues, as well as from cancer cells under unfavorable microenvironments to expedite cancer cell epithelial-to-mesenchymal transition (EMT), migration, invasion, and metastasis. See Li W, Li Y, Guan S, Fan J, Cheng C-F, Bright A M, et al. Extracellular heat shock protein-90α: linking hypoxia to skin cell motility and wound healing. EMBO J 2007;26(5):1221-33; Xu A, Tian T, Hao J, Liu J, Zhang Z, Hao J, et al. Elevation of serum HSP90α correlated with the clinical stage of non-small cell lung cancer. J Cancer Mol 2007;3(4):107-12; and Wang X, Song X, Zhuo W, Fu Y, Shi H, Liang Y, et al. The regulatory mechanism of HSP90α secretion and its function in tumor malignancy. Proc Natl Acad Sci USA 2009;106(50):21288-93. Clinically, elevation of serum/plasma HSP90α levels has been detected from several malignancies including CRC and PDAC. See Wang X, Song X, Zhuo W, Fu Y, Shi H, Liang Y, et al. The regulatory mechanism of HSP90α secretion and its function in tumor malignancy. Proc Natl Acad Sci USA 2009;106(50):21288-9; Wang X, Song X, Zhuo W, Fu Y, Shi H, Liang Y, et al. The regulatory mechanism of HSP90α secretion and its function in tumor malignancy. Proc Natl Acad Sci USA 2009;106(50):21288-93; Chen J S, Hsu Y M, Chen C C, Chen L L, Lee C C, Huang T S. Secreted heat shock protein 90α induces colorectal cancer cell invasion through CD91/LRP-1 and NF-κB-mediated integrin α_{V} expression. J Biol Chem 2010;285(33):25458-66; and Chen C C, Chen L L, Li C P, Hsu Y T, Jiang S S, Fan C S, et al. Myeloid-derived macrophages and secreted HSP90α induce pancreatic ductal adenocarcinoma development. OncoImmunology 2018;7(5):e1424612. Elevated levels of such extracellular HSP90α (eHSP90α) can also be detected from pancreatitis patients and PDAC-developing activated K-Ras knock-in mice. See Chen C C, Chen L L, Li C P, Hsu Y T, Jiang S S, Fan C S, et al. Myeloid-derived macrophages and secreted HSP90α induce pancreatic ductal adenocarcinoma development. OncoImmunology 2018;7(5):e1424612. eHSP90α can be produced from pancreas-infiltrating myeloid-derived macrophages and the stimulated pancreatic ductal epithelial cells to promote the macrophage-associated PDAC development. See Chen C C, Chen L L, Li C P, Hsu Y T, Jiang S S, Fan C S, et al. Myeloid-derived macrophages and secreted HSP90α induce pancreatic ductal adenocarcinoma development. OncoImmunology 2018;7(5):e1424612. Additionally, HSP90α secreted by EndoMT-derived CAFs or recombinant HSP90α (rHSP90α) is able to induce M2-marker expression and a feedforward loop of HSP90α secretion from macrophages, which can account for why M2-polarized macrophages cause not only an immunosuppressive and proangiogenic but also an eHSP90α-rich microenvironment to enhance PDAC tumor growth and malignant progression. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138. Altogether, eHSP90α plays critical roles in both tumor development and malignant progression and can be considered as an important therapeutic target.

In our previous study, we synthesized a cell-impermeable small-molecule HSP90α inhibitor to target eHSP90α. See Chen C C, Chen L L, Li C P, Hsu Y T, Jiang S S, Fan C S, et al. Myeloid-derived macrophages and secreted HSP90α induce pancreatic ductal adenocarcinoma development. OncoImmunology 2018;7(5):e1424612. Although it exhibited some inhibitory levels in PDAC cell tumorigeneity and metastasis, it was easy to be excreted out of mouse bodies and the frequent administration caused mouse splenic enlargement. On the other hand, we got a hopeful clue from using anti-HSP90α mouse monoclonal antibody. Our recent studies revealed that anti-HSP90α antibody exhibited a potent therapeutic efficacy against the EndoMT-promoted and M2-macrophage-involved PDAC tumor growth. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138. Anti-HSP90α antibody could block the ligation of eHSP90α with its cell-surface receptor CD91 and in turn prevented eHSP90α-induced a feedforward loop of eHSP90α expression and secretion. This finding has been supported by our another recent study. Octyl gallate (OG), a common antioxidant and preservative safely used in food additive and cosmetics, also showed an inhibitory effect on EndoMT-derived CAFs and eHSP90α-induced macrophage M2-polarization and more HSP90α expression through the blocking of eHSP90α—TLR4 ligation. See Chua K V, Fan C S, Chen L L, Chen C C, Hsieh S C, Huang T S. Octyl gallate induces pancreatic ductal adenocarcinoma cell apoptosis and suppresses endothelial-mesenchymal transition-promoted M2-macrophages, HSP90α secretion, and tumor growth. Cells 2020;9(1):91.

Taken all together, eHSP90α is a potential therapeutic target for desmoplastic and M2-macrophage-exacerbated cancers and development of anti-HSP90α antibody can be a valuable and hopeful strategy to target eHSP90α.

### SUMMARY

In one aspect, described herein is an isolated antibody. The isolated antibody comprises: novel complementarity determining regions (CDRs) capable of specifically binding to the HSP90α epitope containing the amino acid sequence EDK in the amino acid 235 to 244 and amino acid 251 to 260 regions.

In some embodiment, the HSP90α may be eHSP90α.

In some embodiments, the isolated antibody may comprise: heavy chain complementary determining regions CDR1, CDR2 and CDR3 of a heavy chain variable region sequence of SEQ ID NO: 2 or SEQ ID NO: 12; and light chain complementary determining regions CDR1, CDR2 and CDR3 of a light chain variable region sequence of SEQ ID NO: 7 or SEQ ID NO: 17.

In some embodiments, the heavy chain CDR1, CDR2 and CDR3 are from SEQ ID NO: 2, and the light chain CDR1, CDR2 and CDR3 may be from SEQ ID NO: 7.

In some embodiments, the isolated antibody may include a heavy chain variable region that is at least 80% (e.g. 85%, 90%, 95%, 96%, 97%, 98%, 99% or 100%) identical to the sequence of SEQ ID NO: 2, and a light chain variable region that is at least 80% (e.g. 85%, 90%, 95%, 96%, 97%, 98%, 99% or 100%) identical to the sequence of SEQ ID NO: 7.

In some embodiments, the heavy chain CDR1 may have the sequence of SEQ ID NO: 3, the heavy chain CDR2 may have the sequence of SEQ ID NO: 4, the heavy chain CDR3 may have the sequence of SEQ ID NO: 5, the light chain CDR1 may have the sequence of SEQ ID NO: 8, the light chain CDR2 may have the sequence of SEQ ID NO: 9, and the light chain CDR3 may have the sequence of SEQ ID NO: 10.

In some embodiments, the heavy chain CDR1, CDR2 and CDR3 may be from SEQ ID NO: 12, and the light chain CDR1, CDR2 and CDR3 are from SEQ ID NO: 17.

In some embodiments, the isolated antibody may include a heavy chain variable region that is at least 80% (e.g. 85%, 90%, 95%, 96%, 97%, 98%, 99% or 100%) identical to the sequence of SEQ ID NO: 12, and a light chain variable region that is at least 80% (e.g. 85%, 90%, 95%, 96%, 97%, 98%, 99% or 100%) identical to the sequence of SEQ ID NO: 17.

In some embodiments, the heavy chain CDR1 may have the sequence of SEQ ID NO: 13, the heavy chain CDR2 may have the sequence of SEQ ID NO: 14, the heavy chain CDR3 may have the sequence of SEQ ID NO: 15, the light chain CDR1 may have the sequence of SEQ ID NO: 18, the light chain CDR2 may have the sequence of SEQ ID NO: 19, and the light chain CDR3 may have the sequence of SEQ ID NO: 20.

In some embodiments, the isolated antibody may be an antibody containing an Fc region, an Fab fragment, an Fab′ fragment, an F(ab′)2 fragment, a single-chain antibody, an scFV multimer, a monoclonal antibody, a monovalent antibody, a multispecific antibody, a humanized antibody, or a chimeric antibody.

Also described herein are nucleic acid molecules containing nucleic acid sequences that encode the antibody disclosed herein.

In some embodments, provided herein is a host cell that containins the nucleic acid molecules.

Also described herein is a pharmaceutical composition containing the antibody described herein or the nucleic acid molecules containing the nucleic acid sequences that encode the antibody disclosed herein. The pharmaceutical composition may further comprise a pharmaceutically acceptable carrier and/or another therapeutic agent (e.g., another cancer drug, a cytotoxic agent or an immunomodulatory). Examples of the therapeutic agent may include, but are not limited to, gemcitabine.

eHSP9060 may be used as a therapeutic strategy for desmoplasia (in particular, cancer desmoplasia), thus, also described herein is a method for treating cancer desmoplasia in a subject, comprising: administering a therapeutic agent targeting eHSP90α to the subject in need thereof.

In some embodiment, the antibody described herein may also be used to inhibit cancer cell growth or cancer cell metastasis. Thus, described herein is a method for treating a cancer in a subject, comprising: administering to the subject in need thereof an effective amount of the antibody described herein or the nucleic acid molecules containing the nucleic acid sequences that encode the antibody disclosed herein.

In some embodiment, the antibody described herein may also be used to reduce desmoplasia or prevent the formation of desmoplasia. Thus, described herein is a method for treating desmoplasia in a subject, comprising: administering to the subject in need thereof an effective amount of the antibody described herein or the nucleic acid molecules containing the nucleic acid sequences that encode the antibody disclosed herein. Herein, desmoplasia may be cancer desmoplasia.

In some embodiment, the method may further include, administering another therapeutic agent to the subject. The time for administering the therapeutic agent is not particularly limited. In some embodiment, the therapeutic agent may be administered when administering the antibody or the nucleic acid molecules. In some embodiment, the therapeutic agent may be administered after administering the antibody or the nucleic acid molecules.

In some embodiment, the blood HSP90α level in the subject may be detected using the isolated antibody described herein to monitor the shrink of tumor in the subject in the method for treating the cancer or desmoplasia in the subject. In some embodiment, the blood HSP90α level in the subject may be the blood HSP90α level in the whole blood or serum of the subject.

In some embodiments, the cancer may have a desmoplasia feature.

In some embodiments, the cancer may have a M2-macrophage-exacerbated feature.

Examples of the cancer may include, but are not limited to pancreatic cancer, colon cancer, breast cancer, liver cancer or lung cancer.

Also provided herein is a method for evaluating the shrink of tumor in a subject, comprising: obtaining a blood sample of the subject; and determing a HSP90α level in the blood sample. In some embodiments, the blood sample may be whole blood or serum.

In some embodiments, the method for evaluating the shrink of tumor in a subject may comprise: obtaining a blood sample of a subject administered with IgG and another blood sample of another subject administered with the antibody described herein or the nucleic acid molecules containing the nucleic acid sequences that encode the antibody disclosed herein; determing HSP90α levels in the blood samples of the subjects administered with IgG, the antibody, or the nucleic acid molecules described herein. When the HSP90α level in the blood sample of the subject administered with the antibody or the nucleic acid molecules disclosed herein is less than the HSP90α level in the blood sample of the subject administered with IgG, it indicates that the antibody or the nucleic acid molecules described herein can effectively inhibit the growth of the tumor or reduce the tumor volume.

The details of one or more embodiments of the disclosure are set forth in the description below. Other features, objects, and advantages of the disclosure will be apparent from the description and from the claims.

### BRIEF DESCRIPTION OF DRAWINGS

FIG. 1 is a set of graphs showing characterization of mouse anti-HSP90α monoclonal antibodies. (a) Western blotting analysis for the HSP90α-binding ability of the culture supernatants of six hybridoma clones derived from the mice immunized with rHSP90α. (b) ELISA for the HSP90α-binding activities of the culture supernatants of six hybridoma clones. (c) Subtype characterization for identifying both Clone-2 and Clone-6 antibodies as IgG2b-isotype immunoglobulins containing κ light chains. (d) Proximity ligation assay (PLA) for the levels of physical associations of CD91 with HSP90α and IKKα, respectively, in PANC-1 cells stimulated with 15 μg/ml of rHSP90α in the absence or presence of 10 μg/ml of control IgG or Clone-2 or Clone-6 anti-HSP90α antibody. (e) Transwell invasion assay for cellular invasion abilities of SW620, LoVo, and BxPC-3 cells pre-treated 16 h with PBS or 15 μg/ml of rHSP90α. (f) Transwell invasion assay for cellular invasion abilities of SW620 and PANC-1 cells pre-stimulated 16 h with 15 μg/ml of rHSP90α plus 10 μg/ml of control IgG or Clone-2 or Clone-6 anti-HSP90α antibody. (g) Assay for cellular spheroid-forming abilities of SW620, PANC-1, and Panc 02 cells pre-treated 16 h with 15 μg/ml of rHSP90α plus 10 μg/ml of control IgG or Clone-2 or Clone-6 anti-HSP90α antibody.

FIG. 2 is a set of graphs showing characterization of humanized anti-HSP90α antibodies. (a) Assay for HSP90α-binding kinetics of Clone-2-chimera, Clone-2-hA, Clone-2-hB, and Clone-2-hC anti-HSP90α antibodies using Biacore T200. (b) The equilibrium dissociation constant (K_{D}) values of Clone-2-chimera, Clone-2-hA, Clone-2-hB, and Clone-2-hC antibodies. The binding sensor grams were generated using Biacore T200 and then fitted to a simple 1:1 interaction model. (c) Transwell invasion assay for cellular invasion abilities of PANC-1 cells pre-treated 16 h with 15 μg/ml of rHSP90α plus 10 μg/ml of control IgG or Clone-2-chimera, Clone-2-hA, Clone-2-hB, or Clone-2-hC anti-HSP90α antibody. (d) Assay for cellular spheroid-forming abilities of PANC-1 cells pre-treated 16 h with 15 μg/ml of rHSP90α plus 10 μg/ml of control IgG or Clone-2-chimera, Clone-2-hA, Clone-2-hB, or Clone-2-hC anti-HSP90α antibody. (e) Viabilities of ARPE-19 retinal pigmented epithelial cells upon 72-h treatments with series concentrations of 17-AAG and HH01 (i.e., Clone-2-hA) antibody, respectively. (f) Pharmacokinetics of HH01 antibody in male mice after intravenous (IV) injection with a single dose of 10 mg/kg.

FIG. 3 is a set of graphs showing identification of the epitopes of Clone-2-chimera and HH01 (i.e., Clone-2-hA) anti-HSP90α antibodies. (a) Domain scanning assay to delineate which HSP90α region was responsible for the binding with Clone-2 antibody. (b) Peptide scanning assay to investigate which sites on HSP90α were responsible for the binding with Clone-2-chimera antibody. (c) Peptide scanning assay to investigate which sites on HSP90α were responsible for the binding with HH01 antibody. (d) Alanine scanning assay to determine which a.a. residues of HSP90α were critical for the binding with Clone-2-chimera and HH01 antibodies. (e) Transwell invasion assay for cellular invasion abilities of PANC-1 cells pre-treated 16 h with 15 μg/ml of rHSP90α plus 15 μg/ml of control peptide (P-ctrl) or epitopes-containing peptide (P-46). (f) Assay for cellular spheroid-forming abilities of PANC-1 cells pre-treated 16 h with 15 μg/ml of rHSP90α plus 15 μg/ml of control peptide (P-ctrl) or epitopes-containing peptide (P-46).

FIG. 4 is a set of graphs showing evaluation of the prophylactic efficacy of HH01 antibody in desmoplastic mouse PDAC model. (a) Masson trichrome staining assay to reveal tumor desmoplasia in the EndoMT cells-involved Panc 02 tumor which was not observed in the tumor derived from Panc 02 cells alone. (b) A schematic illustration showing the prophylactic HH01 administration schedule in our desmoplastic mouse PDAC model. (c) Measurement of superficial tumor volumes to plot tumor growth curves of EndoMT-involved Panc 02 cell grafts from the mice treated with control IgG and HH01 antibody, respectively. (d) Tumor weights obtained from the sacrificed mice on Day-30 post-inoculation. (e) Masson trichrome staining of the tumor tissue sections from the mice treated with control IgG or HH01 antibody. (f) Serum HSP90α levels of the mice subcutaneously inoculated with Panc 02 plus EndoMT cell grafts and treated with control IgG or HH01 antibody.

FIG. 5 is a set of graphs showing evaluation of the therapeutic efficacy of HH01 antibody in desmoplastic mouse PDAC model. (a) A schematic illustration showing the therapeutic HH01 administration schedule in our desmoplastic mouse PDAC model. (b) Measurement of superficial tumor volumes to plot tumor growth curves of EndoMT-involved Panc 02 cell grafts from the mice treated with control IgG and HH01 antibody, respectively. (c) Tumor weights obtained from the sacrificed mice on Day-41 post-inoculation. (d) Masson trichrome staining of the tumor sections from the mice treated with control IgG or HH01 antibody. (e) Serum HSP90α levels of the mice subcutaneously inoculated with Panc 02 plus EndoMT cell grafts and treated with control IgG or HH01 antibody.

FIG. 6 is a set of graphs showing evaluation of the therapeutic efficacy of HH01 antibody in desmoplastic humanized mouse PDAC model. (a) A schematic illustration showing the therapeutic HH01 administration schedule in our desmoplastic humanized mouse PDAC model. (b) Measurement of superficial tumor volumes to plot tumor growth curves of EndoMT-involved PANC-1 cell grafts from the mice treated with control IgG, control IgG plus gemcitabine, and HH01 antibody plus gemcitabine, respectively. (c) Tumor weights obtained from the sacrificed mice on Day-39 post-inoculation. (d) Masson trichrome staining of the tumor sections from the mice with different treatments. (e) Serum HSP90α levels of the mice subcutaneously inoculated with PANC-1 plus EndoMT cell grafts and treated with control IgG, control IgG plus gemcitabine, or HH01 antibody plus gemcitabine.

FIG. 7 is a set of graphs showing evaluation of the therapeutic efficacy of HH01 antibody in K-Ras^{G12D}-induced desmoplastic mouse PDAC model. (a) A schematic illustration showing the therapeutic HH01 administration schedule in K-Ras^{G12D}-induced desmoplastic mouse PDAC model. (b) Kaplan-Meier survival curves of the LSL-KrasG12D/Pdx1-Cre mice treated with control IgG and HH01 antibody, respectively. (c) Example pancreases and livers and their desmoplasia levels from the LSL-KrasG12D/Pdx1-Cre mice treated with control IgG or HH01 antibody. (d) Serum HSP90α levels of the LSL-KrasG12D/Pdx1-Cre mice treated with control IgG or HH01 antibody.

FIG. 8 is a set of graphs showing eHSP90α-induced M2-type macrophages exhibiting significant tumor-promoting activity. (a & b) Promotion of the tumor growth of Panc 02 cell grafts by rHSP90α-treated macrophages. C57BL/6 mice were subcutaneously injected with Panc 02 cells alone or together with the BMDMs pretreated 24 h with 100 ng/ml of LPS or 15 μg/ml of rHSP90α (n=6 per group). The sizes of developing tumors were superficially measured using a Vernier caliper and their volumes were calculated with the formula of ½×length×width² (a). Mice were sacrificed on Day 28 post-inoculation and tumors were removed (b). ^{@}P<0.01 when compared with “Panc 02” or “Panc 02+BMDM”. (c) Immunohistochemical staining analyses of F4/80, CD163, CD4, and CD8 from the tumors formed by Panc 02 cells alone (designated as “Panc 02” group) or Panc 02 cells mixed with PBS-, LPS-, or rHSP90α-treated BMDMs (designated as “Panc 02+BMDM”, “Panc 02+LPS-treated BMDM”, and “Panc 02+rHSP90α-treated BMDM” groups, respectively). H/E, Hematoxylin and Eosin staining. (d) DAPI-stained tissue sections of the tumors derived from “Panc 02”, “Panc 02+BMDM”, “Panc 02+LPS-treated BMDM”, and “Panc 02+rHSP90α-treated BMDM” cell grafts.

FIG. 9 is a set of graphs showing evaluation of the therapeutic efficacy of HH01 antibody in M2-macrophage-exacerbated mouse PDAC model. (a) A schematic illustration showing the therapeutic HH01 administration schedule in our M2-macrophage-exacerbated mouse PDAC model. (b) Measurement of superficial tumor volumes to plot tumor growth curves of M2-macrophage-involved Panc 02 cell grafts from the mice treated with control IgG and HH01 antibody, respectively. (c) Tumor weights obtained from the sacrificed mice on Day-42 post-inoculation. (d) Serum HSP90α levels of the mice subcutaneously inoculated with Panc 02 plus M2-macrophage cell grafts and treated with control IgG or HH01 antibody. (e) Immunohistochemical staining analyses of CD163, CD204, CD4, and CD8 from the tumors of the mice treated with control IgG and HH01 antibody. H/E, Hematoxylin and Eosin staining.

FIG. 10 is a set of graphs showing immunohistofluorescent staining analyses of F4/80, iNOS, Arginase 1, CD4, CD8, and TNF-α from the tumors of the mice treated with control IgG and HH01 antibody. (a & b) Increase of F4/80⁺iNOS⁺ cells (indicated by arrows) and decrease of F4/80⁺Arginase1⁺ cells (indicated by arrows) were observed from the tumor tissues of HH01-treated mice. (c) Increase of CD4⁺TNF-α⁺ cells (indicated by arrows) was observed from the tumor tissues of HH01-treated mice. (d) Increase of CD8⁺TNF-α⁺ cells (indicated by white arrows) was observed from the tumor tissues of HH01-treated mice.

### DETAILED DESCRIPTION

Described herein are novel antibodies that bind to HSP90α, such as eHSP90α.

The isolated antibody may include novel CDRs capable of specifically binding to the HSP90α epitope containing the amino acid sequence EDK in the ₂₃₅AEEKEDKEEE₂₄₄ and ₂₅₁ESEDKPEIED₂₆₀ regions.

The isolated antibody may comprise: heavy chain complementary determining regions CDR1, CDR2 and CDR3 of a heavy chain variable region sequence of SEQ ID NO: 2 or SEQ ID NO: 12; and light chain complementary determining regions CDR1, CDR2 and CDR3 of a light chain variable region sequence of SEQ ID NO: 7 or SEQ ID NO: 17.

In one aspect, the heavy chain CDR1, CDR2 and CDR3 may be from SEQ ID NO: 2, and the light chain CDR1, CDR2 and CDR3 may be from SEQ ID NO: 7. In some embodiments, the isolated antibody may include a heavy chain variable region that is at least 80% (e.g. 85%, 90%, 95%, 96%, 97%, 98%, 99% or 100%) identical to the sequence of SEQ ID NO: 2, and a light chain variable region that may be at least 80% (e.g. 85%, 90%, 95%, 96%, 97%, 98%, 99% or 100%) identical to the sequence of SEQ ID NO: 7. In some embodiments, the heavy chain CDR1 may have the sequence of SEQ ID NO: 3, the heavy chain CDR2 may have the sequence of SEQ ID NO: 4, the heavy chain CDR3 may have the sequence of SEQ ID NO: 5, the light chain CDR1 may have the sequence of SEQ ID NO: 8, the light chain CDR2 may have the sequence of SEQ ID NO: 9, and the light chain CDR3 may have the sequence of SEQ ID NO: 10. In some embodiments, nucleic acid molecules containing nucleic acid sequences that encode the antibody includes a heavy chain variable region that is at least 80% identical to the sequence of SEQ ID NO: 2, and a light chain variable region that is at least 80% identical to the sequence of SEQ ID NO: 7 may also be provided. In some embodiments, the nucleic acid molecules may include a sequence that is at least 80% (e.g. 85%, 90%, 95%, 96%, 97%, 98%, 99% or 100%) identical to the sequence of SEQ ID NO: 1 for encoding the heavy chain variable region, and a sequence that is at least 80% (e.g. 85%, 90%, 95%, 96%, 97%, 98%, 99% or 100%) identical to the sequence of SEQ ID NO: 6 for encoding the light chain variable region.

The sequences of SEQ ID NO: 1 to SEQ ID NO: 10 are listed in the following Table 1.

TABLE 1
Clone-2 anti-HSP90α antibody
Heavy chain variable region

| | |
|---|---|
| DNA sequence | AGGTGCAGGTTGTTGAGTCTGGTGGAGGATTGGTGCAGCCTAAAG |
| SEQ ID NO: 1 | GGTCATTGAAACTCTCATGTGCAGCCTCTGGATTCACCTTCAAAAC |
| | CTACGCCATGAACTGGGTCCGCCAGGCTCCAGGAAAGGGTTTGGA |
| | ATGGGTTGCTCGCATAAGAAGTAAAAGTAGAAATTATGCAACATA |
| | TTATGCCGATTCAGTGAAAGACAGGTTCACCATCTCCAGAGATGAT |
| | TCACAATTCATGTTCTATCTGCAAATGAACAACTTGAAAACTGAGG |
| | ACACAGCCATGTATTACTGTGTTGTGGGGGGCTCGGGTTTTGACTA |
| | CTGGGGCCAAGGCACCACTCTCACAGTCTCCTCA |
| Amino acid | EVQVVESGGGLVQPKGSLKLSCAASGFTFKTYAMNWVRQAPGKGL |
| sequence | EWVARIRSKSRNYATYYADSVKDRFTISRDDSQFMFYLQMNNLKTE |
| SEQ ID NO: 2 | DTAMYYCVVGGSGFDYWGQGTTLTVSS |
| CDR1 | GFTFKTYA |
| SEQ ID NO: 3 | |
| CDR2 | IRSKSRNYAT |
| SEQ ID NO: 4 | |
| CDR3 | VVGGSGFDY |
| SEQ ID NO: 5 | |

Light chain variable region

| | |
|---|---|
| DNA sequence | GATGTTTTGATGACCCAAACTCCACTCTCCCTGCCTGTCAGTCTTGG |
| SEQ ID NO: 6 | AGATCAAGCCTCCATCTCTTGCAGATCTAGTCAGAGCATTGTACAT |
| | AGTAATGGAAACACCTATTTAGAATGGTACCTGCAGAAACCAGGC |
| | CAGTCTCCAAAGCTCCTGATCTACAAAGTTTCCAACCGATTTTCTG |
| | GGGTCCCAGACAGGTTCAGTGGCAGTGGATCAGGGACAGATTTCA |
| | CACTCAGCATCAGCAGAGTGGAGGCTGGGGATCTGGGAGTTTATT |
| | ACTGCTTTCAAGGTTCACATGTTCCGTGGACGTTCGGTGGAGGCAC |
| | CAAGCTGGAAATCAAA |
| Amino acid | DVLMTQTPLSLPVSLGDQASISCRSSQSIVHSNGNTYLEWYLQKPGQ |
| sequence | SPKLLIYKVGVPDRFSGSGSGTDFTLSISRVEAGDLGVYYCFQGSHVP |
| SEQ ID NO: 7 | WTFGGGTKLEIK |
| CDR1 | RSSQSIVHSNGNTYLE |
| SEQ ID NO: 8 | |
| CDR2 | KVSNRFS |
| SEQ ID NO: 9 | |
| CDR3 | FQGSHVPWT |
| SEQ ID NO: 10 | |

Bold faced: CDR

In one aspect, the heavy chain CDR1, CDR2 and CDR3 may be from SEQ ID NO: 12, and the light chain CDR1, CDR2 and CDR3 may be from SEQ ID NO: 17. In some embodiments, the isolated antibody may include a heavy chain variable region that is at least 80% (e.g. 85%, 90%, 95%, 96%, 97%, 98%, 99% or 100%) identical to the sequence of SEQ ID NO: 12, and a light chain variable region that may be at least 80% (e.g. 85%, 90%, 95%, 96%, 97%, 98%, 99% or 100%) identical to the sequence of SEQ ID NO: 17. In some embodiments, the heavy chain CDR1 may have the sequence of SEQ ID NO: 13, the heavy chain CDR2 may have the sequence of SEQ ID NO: 14, the heavy chain CDR3 may have the sequence of SEQ ID NO: 15, the light chain CDR1 may have the sequence of SEQ ID NO: 18, the light chain CDR2 may have the sequence of SEQ ID NO: 19,and the light chain CDR3 may have the sequence of SEQ ID NO: 20. In some embodiments, nucleic acid molecules containing nucleic acid sequences that encode the antibody includes a heavy chain variable region that is at least 80% identical to the sequence of SEQ ID NO: 12, and a light chain variable region that is at least 80% identical to the sequence of SEQ ID NO: 17 may also be provided. In some embodiments, the nucleic acid molecules may include a sequence that is at least 80% (e.g. 85%, 90%, 95%, 96%, 97%, 98%, 99% or 100%) identical to the sequence of SEQ ID NO: 11 for encoding the heavy chain variable region, and a sequence that is at least 80% (e.g. 85%, 90%, 95%, 96%, 97%, 98%, 99% or 100%) identical to the sequence of SEQ ID NO: 16 for encoding the light chain variable region.

The sequences of SEQ ID NO: 11 to SEQ ID NO: 20 are listed in the following Table 2.

TABLE 2
HH01 anti-HSP90α antibody
Heavy chain variable region

| | |
|---|---|
| DNA sequence | GAGGTGCAGCTGGTGGAGTCTGGAGGAGGACTGGTGCAGCCAGGA |
| SEQ ID NO: 11 | GGAAGCCTGAGACTGTCTTGCGCCGCTTCCGGCTTCACCTTTAAGA |
| | CATACGCCATGAACTGGGTGAGGCAGGCTCCTGGACAGGGACTGG |
| | AGTGGGTGGCTAGGATCCGGTCTAAGTCCCGGAATTACGCCACCTA |
| | CTATGCTGACTCCGTGAAGGATAGATTCACAATCAGCCGCGACGATT |
| | CTAAGAACCAGTTTTATCTGCAGATGAACAATCTGCGCACCGAGGA |
| | CACAGCCGTGTACTATTGCGTGGTGGGCGGCAGCGGCTTCGATTATT |
| | GGGGCCAGGGCACCACACTGACCGTGTCCAGC |
| Amino acid | EVQLVESGGGLVQPGGSLRLSCAASGFTFKTYAMNWVRQAPGQGLE |
| sequence | WVARIRSKSRNYATYYADSVKDRFTISRDDSKNQFYLQMNNLRTEDT |
| SEQ ID NO: 12 | AVYYCVVGGSGFDYWGQGTTLTVSS |
| CDR1 | TFKTYAMN |
| SEQ ID NO: 13 | |
| CDR2 | RIRSKSRNYATYYADSVKD |
| SEQ ID NO: 14 | |
| CDR3 | GSGFDY |
| SEQ ID NO: 15 | |

Light chain variable region

| | |
|---|---|
| DNA sequence | GACGTGGTCATGACCCAGACACCACTGTCCCTGCCTGTGAGCCTGG |
| SEQ ID NO: 16 | GCGATCAGGCCAGCATCTCTTGCAGGTCCAGCCAGTCTATCGTGCA |
| | CTCCAACGGCAATACCTACCTGGAGTGGTATCTGCAGAAGCCAGGC |
| | CAGTCCCCCCAGCTGCTGATCTACAAGGTGAGCAACAGGTTCTCTG |
| | GAGTGCCTGACCGGTTTTCCGGAAGCGGATCTGGCACAGATTTCAC |
| | CCTGACAATCTCTTCCGTGGAGGCTGAGGACCTGGGCGTGTACTATT |
| | GTTTCCAGGGCTCCCATGTGCCATGGACCTTTGGCGGCGGCACAAA |
| | GCTGGAGATCAAG |
| Amino acid | DVVMTQTPLSLPVSLGDQASISCRSSQSIVHSNGNTYLEWYLQKPGQ |
| sequence | SPQLLIYKVSNRFSGVPDRFSGSGSGTDFTLTISSVEAEDLGVYYCFQG |
| SEQ ID NO: 17 | SHVPWTFGGGTKLEIK |
| CDR1 | RSSQSIVHSNGNTYYLE |
| SEQ ID NO: 18 | |
| CDR2 | KVSNRFS |
| SEQ ID NO:19 | |
| CDR3 | FQGSHVP |
| SEQ ID NO: 20 | |

Bold faced: CDR

The antibody can bind specifically to HSP90α. More specifically, the antibody can bind to HSP90α with a higher affinity than other non-HSP90α proteins. In addition, the CDRs of the heavy or light chain variable region can be determined by any method known in the art.

The antibody described herein exhibits a high bidning affinity toward eHSP90α. Thus, the antibody described herein can inhibit the desmoplasia within tumor, to further suppress the tumor growth or decrease the tumor size.

Based on the sequence of the antibody disclosed herein and their CDRs, a skilled person may produce an anti-HSP90α antibody in various forms using any method know in the art, and the produced anti-HSP90α antibody can specifically bind to the HSP90α epitope containing two EDK sites in the amino acid 235 to 244 and amino acid 251 to 260 regions.

Based on the sequence of HSP90α epitope, a synthesized peptide may competitively suppress the protumor functions of eHSP90α.

The term “antibody” used herein includes various antibody structures with the antigen-binding activity. For example, the antibody may include, but is not limited to, an antibody containing an Fc region, an Fab fragment, an Fab′ fragment, an F(ab′)2 fragment, a single-chain antibody, an scFV multimer, a monoclonal antibody, a monovalent antibody, a multispecific antibody, a humanized antibody, or a chimeric antibody. In some embodiments, the antibody is a humanized antibody.

Also described herein is a pharmaceutical composition containing the antibody described herein. The pharmaceutical composition comprises: the isolated antibody described herein and a pharmaceutically acceptable carrier.

Also described herein is a pharmaceutical composition containing the nucleic acid molecules capable of encoding the antibody described herein. The pharmaceutical composition comprises: the nucleic acid molecules capable of encoding the antibody described herein and a pharmaceutically acceptable carrier.

The term “pharmaceutically acceptable carrier” means that the carrier must be compatible with the active ingredients (for example, capable of stabilizing the antibody) and not be deleterious to the subject to be treated. The carrier may be at least one selected from the group consisting of active agents, adjuvants, dispersants, wetting agents and suspending agents. The example of the carrier may be, but is not limited to, microcrystalline cellulose, mannitol, glucose, non-fat milk powder, polyethylene, polyvinylprrolidone, starch or a combination thereof.

The antibody, the nucleic acid molecules or the pharmaceutical composition containing one or more of them can be administered to a subject orally, parenterally, by inhalation spray, topically, rectally, nasally, buccally, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

Also described herein is use of the antibody or the nucleic acid molecules for the manufacture of a medicament for treating a cancer.

Also described herein is a method for treating a cancer in a subject, comprising: administering to the subject an effective amount of the antibody or the nucleic acid molecules described herein.

Examples of the cancer may include, but is not limited to, bladder cancer, bone cancer, brain cancer, breast cancer, cervical caner, colon cancer, endometrial cancer, esophageal cancer, leukemia, liver cancer, lymphoma, kidney cancer, osteosarcoma, ovarian cancer, pancreatic cancer, prostate cancer, skin cancer including basal and squamous cell carcinoma and melanoma, small intestine cancer, stomach cancer, thymus cancer and thyroid cancer. In some embodiments, the cancer has a desmoplasia feature, and in particular, the desmoplasia can be found within or around tumor cell. In some embodiments, the cancer may be pancreatic cancer, colon cancer, breast cancer, liver cancer or lung cancer.

The antibody described herein may inhibit cancer desmoplasia, immunosuppression, growth, and metastasis.

The term “subject” refers to human or non-human animal.

The term “treating”, “treat” or “treatment” refers to application or administration of the antibody, nucleic acid molecules or pharmaceutical composition to a subject with the purpose to cure, alleviate, relieve, alter, remedy, improve, or affect the disease, the symptom, or the predisposition. “An effective amount” refers to the amount of the antibody or nucleic acid molecules which is required to confer the desired effect on the subject. Effective amounts vary, as recognized by those skilled in the art, depending on route of administration, excipient usage, and the possibility of co-usage with other therapeutic treatments such as use of other active agents.

The details of one or more embodiments of the disclosure are set forth in the description below. Other features, objects, and advantages of the disclosure will be apparent from the description and from the claims.

### EXAMPLE

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever. All publications cited herein are incorporated by reference in their entirety.

### Materials and Methods

Cell cultures

Human PDAC cell line PANC-1 was cultivated in a 37° C. and 5% CO₂ humidified incubator with Dulbecco's Modified Eagle's Medium (DMEM) supplemented with 10% of fetal bovine serum (FBS) and a mix of 100 units/ml of penicillin, 100 μg/ml of streptomycin, and 2 mM of _{L}-glutamine (1×PSG). Human PDAC cell line BxPC-3, human CRC cell line SW620, and mouse PDAC cell line Panc 02 were incubated at 37° C. in an atmosphere of 5% CO₂ and 95% air with RPMI-1640 medium plus 10% FBS and 1×PSG. Under the same growth conditions, human CRC cell line LoVo was maintained in 20% FBS and 1×PSG-containing Ham's F-12 medium, and human retinal pigmented epithelial cell line ARPE-19 (ATCC CRL-2302™; American Type Culture Collection, Manassas, VA, USA) was cultivated in ATCC-formulated DMEM:F12 medium (cat. #30-2006, ATCC) supplemented with 10% FBS and 1×PSG. Human umbilical vein endothelial cells (HUVECs) were isolated and grown in a 37° C. and 5% CO₂ humidified incubator with M199 medium containing 20% FBS, 100 units/ml penicillin, 100 g/ml streptomycin, and 30 μg/ml endothelial cell growth supplement (EMD Millipore, Billerica, MA, USA). See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138. For EndoMT induction, HUVECs were pre-incubated 16 h with 2% FBS-containing M199 medium, and then added with 0.3 μg/ml of OPN and incubated for another 24 h. Mouse endothelial cell line 3B-11 (ATCC CRL-2160™) was grown in RPMI-1640 medium supplemented with 10% FBS and 1×PSG. For EndoMT induction, 3B-11 cells were treated same as HUVECs except for 1% FBS-containing RPMI-1640 medium was used instead. Mouse bone marrow-derived macrophages (BMDMs) were prepared as described previously and cultivated in DMEM plus 10% FBS and 1×PSG. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138.

### Mouse Models

Mouse experiments were performed with the permission of the Institutional Animal Care and Use Committee of National Health Research Institutes (NHRI-IACUC-106031-A, 109022-M2-S02, and 109196-A). To establish a desmoplastic tumor transplant model, C57BL/6 mice at 12 weeks of age were subcutaneously inoculated with a mix of Panc 02 cells and EndoMT-derived CAFs (i.e. OPN-treated 3B-11 cells). See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138; and Chua K V, Fan C S, Chen L L, Chen C C, Hsieh S C, Huang T S. Octyl gallate induces pancreatic ductal adenocarcinoma cell apoptosis and suppresses endothelial-mesenchymal transition-promoted M2-macrophages, HSP90α secretion, and tumor growth. Cells 2020;9(1):91. Further mouse treatments and schedules were as indicated in the figures and the related text. Sizes of developing tumors were superficially measured with a Vernier caliper, and tumor volumes were calculated with the formula ½×length×width². Real tumors were weighed when mice were sacrificed. To establish a desmoplastic tumor transplant model in humanized mice, NOD-SCID IL2R^{null} (ASID) mice transplanted with human hematopoietic stem cells (hHSC) were subcutaneously inoculated with a mix of PANC-1 cells and OPN-treated HUVECs. The employed hHSC-transplanted ASID mice were produced from the National Laboratory Animal Center (Taipei, Taiwan) and all contained human CD45⁺ cell levels more than 38% of total lymphocytes. To establish an M2-macrophage-exacerbated mouse PDAC model, C57BL/6 mice were subcutaneously inoculated with a mix of Panc 02 cells and rHSP90α-treated BMDMs. Further mouse treatments and schedules were as indicated in the figures and the related text. Additionally, LSL-KrasG12D/Pdx1-Cre transgenic mice were adopted as a spontaneously PDAC-developing mouse model to evaluate the therapeutic efficacy of HH01 antibody. LSL-KrasG12D and Pdx1-Cre breeder mice were obtained from the Mouse Models of Human Cancers Consortium Repository of the National Cancer Institute (Frederick, Maryland, USA). LSL-KrasG12D mice were crossed with Pdx1-Cre mice to generate LSL-KrasG12D/Pdx1-Cre mice. The further mouse treatments were performed as indicated in the figure and the related text.

### Western Blot Analysis

Mouse hybridomas were generated from the mice immunized with the recombinant 732-amino acid (a.a.) full-length human HSP90α (rHSP90α; PeproTech Co., Cranbury, NJ, USA) according to the conventional protocol. See Yokoyama W M. Production of monoclonal antibody supernatant and ascites fluid. Curr Protoc Mol Biol 2008;83:11.10.1-11.10.10. Next, the general procedure of western blot analysis was adopted to preliminarily screen the hybridomas. See Fan C S, Chen L L, Hsu T A, et al. Endothelial-mesenchymal transition harnesses HSP90α-secreting M2-macrophages to exacerbate pancreatic ductal adenocarcinoma. J Hematol Oncol 2019;12:138. The culture media of hybridomas were collected to react with the membrane strips containing HSP90α and molecular weight marker proteins blotted from SDS-polyacrylamide gels after electrophoreses. After washed trice with PBS plus 0.05% Tween-20 (PBST), the membrane strips were incubated 1 h with horseradish peroxidase-conjugated secondary antibody. Following three washes with PBST, immunoreactive protein bands were visible after the reaction with 0.3 mg/ml of 3,3′,5,5′-tetramethylbenzidine in 0.015% H₂O₂ (Sigma-Aldrich, St. Louis, MO, USA).

### Enzyme-Linked Immunosorbent Assay (ELISA)

For assaying the HSP90α-binding activities of the prepared antibodies, 96-well plates were coated with 12.5 ng/ml of rHSP90α in the capture buffer (BioLegend, San Diego, CA, USA) by overnight incubation at 4° C. The coated plates were washed twice with PBST and then blocked with PBST plus 3% bovine serum albumin (BSA). The prepared antibodies were applied and incubated 2 h at room temperature. After three washes with PBST, horseradish peroxidase-conjugated secondary antibody was added and incubated at 37° C. for another 1 h. After three washes, 0.3 mg/ml of 3,3′,5,5′-tetramethylbenzidine in 0.015% H₂O₂ was added into each well and incubated in the dark at room temperature for 10 min. The reactions were stopped by the addition of 0.5 M H₂SO₄, and OD₄₅₀ values were measured by an Infinite M200 microplate reader (TECAN, Männedorf, Switzerland). Additionally, the immunoglobulin (IgG) isotype characterization of anti-HSP90α antibodies was performed by using the Mouse Ig Isotyping ELISA Ready-Set-Go!™ Kit (eBioscience™, Thermo Fisher Scientific, Waltham, MA, USA). The ELISA protocol was also used to assay the secreted HSP90α levels of mouse serum samples as described previously. See Chen J S, Hsu Y M, Chen C C, Chen L L, Lee C C, Huang T S. Secreted heat shock protein 90α induces colorectal cancer cell invasion through CD91/LRP-1 and NF-κB-mediated integrin α_{V} expression. J Biol Chem 2010;285(33):25458-66.

### Proximity Ligation Assay (PLA)

PANC-1 cells seeded onto glass coverslips at a density of 2×10⁵ cells per 22×22-mm coverslip were incubated 16 h with 0.5% serum-containing medium at 37° C. plus a 5% CO₂ humidified atmosphere. The cells were then added with PBS or 15 μg/ml of rHSP90α in the absence or presence of 10 μg/ml of control IgG or the tested anti-HSP90α antibody for another 24-h incubation. The treated cells were then fixed with 3% paraformaldehyde and blocked with the blocking solution provided in the Duolink in situ PLA kit (Olink Bioscience, Uppsala, Sweden). Furthermore, the cell samples were incubated overnight at 4° C. with anti-CD91 antibody (1:80, cat. #550495, BD Biosciences, San Jose, CA, USA) mixed with anti-HSP90α antibody (1:80, cat. #AHP-1339, AbD Serotec, Raleigh, NC, USA) or anti-IKKα antibody (1:80, cat. #3285, Epitomics Co., Burlingame, CA, USA). After washed trice with Tris-buffered saline plus 0.05% Tween 20, the cell samples were incubated with PLA probes for the subsequent ligation and amplification procedure according to the manufacturer's instructions for the Duolink in situ PLA kit. Finally, nuclei were counterstained with 4′,6′-diamidino-2-phenylindole (DAPI; Sigma-Aldrich). Images were photographed and analyzed using Leica TCS SP5 II confocal microscope and LAS AF Lite 4.0 software (Leica, Wetzlar, Germany).

### Transwell Invasion Assay

In a 37° C. and 5% CO₂ humidified incubator, cancer cells were subjected to serum starvation in 0.5% FBS-containing medium for 16 h, and then added with PBS or 15 μg/ml of rHSP90α in the absence or presence of 10 μg/ml of control IgG or the tested anti-HSP90α antibody. After another 24 h, the treated cells were harvested as aliquots (1×10⁵ cells per aliquot) in 0.5% FBS-containing culture medium, and then each aliquot was seeded into a top chamber of Transwell inserts pre-coated with 5-fold diluted Matrigel (BD Biosciences). Cells were allowed 16 h to invade through the Matrigel toward the bottom chambers containing culture medium plus 10% FBS. The filters of the Transwell inserts were then fixed and stained with Giemsa. Invasive cells on the lower side of filters were photographed using the Axiovert S100/AxioCam HR microscope system (Carl Zeiss, Oberkochen, Germany) and further quantified by the Image-Pro Plus software (Media Cybernetics, Inc., Silver Spring, MD, USA).

### Cell Spheroid-Forming Assay (Cell Self-Renewal Activity Assay)

One thousand cancer cells were seeded onto each well of 24-well plates pre-coated with a 4-mm thick layer of 0.5% agarose. After incubation 24 h with serum-free medium at 37° C. plus a 5% CO₂ humidified atmosphere, cells were added with PBS or 15 μg/ml of rHSP90α in the absence or presence of 10 μg/ml of control IgG or the tested anti-HSP90α antibody. The incubation was kept for 10-14 days with supplements of some fresh serum-free medium every 3 days. Finally, the cell spheroids characterized by tight, non-adherent, and >100 μm in diameter were photographed and counted under an Olympus IX 71 inverted microscope (Center Valley, PA, USA). The spheroid formation % was calculated by the formula: (number of cell spheroids/1000)×100%.

### Cloning and Sequencing of Antibody Gene From Hybridoma Cellsmouse

Total RNA was isolated from anti-HSP90α IgG-producing mouse hybridoma cells and converted to cDNA by reverse transcriptase plus random hexamers. Mouse Ig-Primer Set (Sigma-Aldrich) was used for the PCR amplification of the variable domains of IgG heavy (H) and light (L) chains (i.e. V_{H} and V_{L} domains). The PCR products were cloned into pJET1.2/blunt cloning vector supplied in the CloneJET PCR cloning kit (Thermo Fisher Scientific) followed by clone selection and DNA sequence analysis.

### Construction and Expression of Recombinant Antibody

The cDNA sequences of the IgG V_{H} and V_{L} domains were synthesized by GeneDireX Inc. (Taoyuan, Taiwan) and inserted into pFUSEss-CHIg-hG1e1 and pFUSE2ss-CLIg-hk plasmids (InvivoGen, San Diego, CA, USA) at the sites just before the regions encoding the constant domains of H and L chains (i.e. C_{H} and C_{L} domains), respectively. For pFUSEss-CHIg-hG1e1 cloning, the V_{H} CDNA was inserted at the EcoR I-Nhe I site behind the human IL-2 signal sequence to express a recombinant protein containing the Fc fragment with mutations at Q250T, E356D, M358L, and L428M residues. On the other hand, the cDNA of the V_{L} domain, after digestion with EcoR I and BsiW I, was inserted into pFUSE2ss-CLIg-hk plasmid. The recombinant DNA sequences encoding the recombinant IgG H and L chains were further amplified using hlgHG-F/hIgHG-R and CLIg-F/CLIg-R primers and then inserted into pcDNA3.4-TOPO plasmid (Thermo Fisher Scientific), respectively. Finally, ExpiCHO cells were co-transfected with the recombinant pcDNA3.4-TOPO plasmids encoding the H chain and L chain by a ratio of 2:3. The media of transfected ExpiCHO cells were harvested after culture for 8-10 days. The recombinant antibody was purified by protein A columns of the Gibco™ ExpiCHO™ Expression System (Thermo Fisher Scientific).

### Antibody Humanization and Optimization

The F (ab′)2 regions of mouse monoclonal antibody were subjected to humanization with the aid of computational methods, and all calculations were performed by Discovery Studio 2018 software (BIOVIA Inc., San Diego, CA, USA). The complementarity determining regions (CDRs) were identified by the published method. See Kabat E A, Wu T T, Perry H M, Gottesman K S, Foeller C. Sequences of proteins of immunological interest. U.S. Department of Health and Human Services, NIH, Bethesda, MD, 1991. The three-dimensional (3D) structures of V_{L} and V_{H} domains were modeling by the X-ray templates of PdbID: 4X0K and PdbID: 4Y5X, respectively. See Johnson J L, Entzminger K C, Hyun J, Kalyoncu S, Heaner D P, Morales Jr I A, Sheppard A, Gumbart J C, Maynard J A, Lieberman R L. Structural and biophysical characterization of an epitope-specific engineered Fab fragment and complexation with membrane proteins: implications for co-crystallization. Acta Crystallogr D Biol Crystallogr 2015;71:896-906; and Moraga I, Wernig G, Wilmes S, Gryshkova V, Richter C P, Hong W J, Sinha R, Guo F, Fabionar H, Wehrman T S, Krutzik P, Demharter S, Plo I, Weissman I L, Minary Majeti P R, Constantinescu S N, Piehler J, Garcia K C. Tuning cytokine receptor signaling by re-orienting dimer geometry with surrogate ligands. Cell 2015;160:1196-1208. The CDRs of mouse antibody were grafted into in-house human templates for further 3D-structure calculations. The calculations included the aspects of aggregation, post-translational modification, protease cleavage, pharmacokinetics, and stability for the mutation suggestions to improve the recombinant antibody.

### HSP90α-Binding Affinity Assay

To measure the HSP90α-binding characteristics of anti-HSP90α antibodies, binding kinetic assays were performed using Biacore T200 (Cytiva Co., Marlborough, MA, USA). The interested anti-HSP90α antibodies were applied onto Protein A Series S sensor chips as ligands to achieve approximately 3000˜5000 response units. A series of concentrations of rHSP90α (1.5625-100 nM) in HBS-EP+ buffer were injected over the chip surface. The association was monitored for 60 sec, and the final dissociation time was 1500 sec.

### Retinal Cell Toxicity Assay

ARPE-19 cells were seeded onto 96-well plates at a density of 4000 cells per well, and then treated in triplicate with various concentrations of 17-AAG or anti-HSP90α antibody HH01 for 72 h. The 3-(4,5-dimethylthiazol-2-yl)-5-(3-carboxymethoxyphenyl)-2-(4-sulfophenyl)-2H-tetrazolium (MTS; Sigma-Aldrich) assay was employed and OD₄₉₀ was measured to evaluate cell viability upon each treatment.

### Pharmacokinetic Assay

The pharmacokinetic profile of anti-HSP90α antibody HH01 was studied in male Bltw:CD1(ICR) male mice (BioLASCO Taiwan Co., Taipei, Taiwan). Mice (n=5) with a body weight of 25-30 g per mouse were intravenously injected with 10 mg/kg of HH01 antibody. Food and water were available ad libitum at all times. For determination of the serum exposure levels of HH01 antibody, blood samples were collected at Day-0.04, 0.08, 0.25, 1, 2, 3, 7, 9, 14, 16, 18, 21, 23, 25, 35, 42, 49, 56, and 63 post administration. These samples were further measured the human IgGI levels by an ELISA kit (Cayman Chemical Co., Michigan, USA) to represent the remaining HH01 levels in mouse sera.

Cloning, Expression, and Purification of Full-Length and Truncated HSP90α

The full-length HSP90α (a.a. 1-732) and its 3 truncated fragments including the N-terminal plus Linker domains (a.a. 1-272), N terminus through Middle domain (a.a. 1-629), and C-terminal domain (a.a. 630-732) were cloned, expressed, and purified. Briefly, each cDNA sequence with a 8×His-tag was amplified by PCR from the human ORF Clone (NM_005348) with the primers containing Xho I and BamH I sites. The PCR products were purified, digested with restriction enzymes, and then inserted into pET-23a plasmid (Sigma-Aldrich). The recombinant plasmids were then transformed into Escherichia coli ClearColi® competent cells (Lucigen Co., Middleton, WI, USA), and expressions of the recombinant constructs were stimulated by the addition of isopropyl-D-thiogalacto-pyranoside. The expressed histidine-tagged full-length and truncated HSP90α were further purified by using the Ni—NTA column (Qiagen, Germantown, MD, USA) according to the manufacturer's instructions. The purified proteins were finally dialyzed against the storage buffer consisting of 25 mM Tris-HCl (pH, 7.0), 50 mM NaCl, 0.1% Triton X-100, 50% glycerol, 1 mM EDTA, and 1 mM DTT, and protein concentrations were determined by the Bradford protein assay (Bio-Rad Laboratories, Hercules, USA).

### Epitope Determination

At first, the domain scanning strategy was used to roughly delineate the HSP90α region responsible for the binding with our prepared antibodies. The above-mentioned full-length HSP90α and its 3 truncated fragments were employed to coat 96-well ELISA plates for assaying the binding activities of the tested mouse monoclonal antibodies. The results suggested that the tested mouse monoclonal antibody bound to the a.a. 1-272 region of HSP90α. Therefore, in peptide scanning strategy, a 132 peptide library was synthesized by Mimotopes Pty. Ltd. (Melbourne, Victoria, Australia), consisting of a series of 10-a.a. peptides offset by 2 a.a. to span the sequence of the a.a. 21-272 region of HSP90α. This series of peptides were used to coat 96-well ELISA plates for assaying the binding activities of the tested antibodies. Based on the sequences suggested by the peptide scanning result, the alanine scanning strategy was further employed to investigate which a.a. residues were critical for the binding of the tested antibodies. The result of peptide scanning assay suggested ₂₃₅AEEKEDKEEE₂₄₄ and ₂₅₁ESEDKPEIED₂₆₀ of HSP90α as the epitopes for binding with the tested antibodies, and therefore 19 peptides were synthesized with sequential substitution of each a.a. by alanine (Genozyme Biotech Inc., Taipei, Taiwan). These peptides were subjected to ELISA as described above.

### Masson Trichrome Staining

Mouse paraffin-embedded tissue sections were deparaffinized by xylene and rehydrated by a series of ethanol dilutions. Masson trichrome staining was performed using a Trichrome Stain Kit (ScyTek Laboratories Inc., Utah, USA) according to the manufacturer's protocol. The stained sections were dehydrated in graded ethanol solutions and xylene, and finally mounted with mounting solution overnight in the dark at room temperature. The sections were observed and photographed using the Axiovert S100/AxioCam HR microscope system.

### Immunohistochemical Staining

Mouse tissue sections with a 4-μm thickness were deparaffinized by xylene and rehydrated in graded ethanol dilutions. Antigen retrieval was performed by heating the sections for 15 min in 10 mM of citrate buffer, pH 6.0, under high pressure. The endogenous peroxidase activity was blocked by 0.3% H₂O₂. Before staining, the sections were blocked 30 min with PBS plus 3% BSA at room temperature in a humidified chamber. Subsequently, anti-F4/80 antibody (1:100, cat. #MCA497R, AbD Serotec), anti-CD163 antibody (1:80, cat. #sc-33560, Santa Cruz Biotechnology, Santa Cruz, CA, USA), anti-CD204 antibody (1:100, cat. #GTX51749, GeneTex Inc., Hsinchu City, Taiwan), anti-CD4 antibody (1:100, cat. #GTX44531, GeneTex Inc.), or anti-CD8 antibody (1:100, cat. #GTX16696, GeneTex Inc.) was applied and incubated at 4° C. for overnight. After washing, the secondary antibodies were applied and incubated 30 min at room temperature. The sections were further detected using the DAKO REAL EnVision Detection System (Produktionsvej 42, DK-2600 Glostrup, Denmark) and counterstained with hematoxylin. The stained sections were dehydrated, mounted with mounting solution, and finally observed and photographed using the Axiovert S100/AxioCam HR microscope system.

### Immunohistofluorescent Staining

Mouse tissue sections were subjected to deparaffinization, rehydration, and antigen retrieval as described above. The sections were blocked 30 min with PBS plus 5% BSA at room temperature in a humidified chamber. Subsequently, 3 antibody sets were applied and incubated overnight at 4° C., respectively. The antibody set-1 was composed of anti-F4/80 antibody (1:100, cat. #MCA497R, AbD Serotec), anti-iNOS antibody (1:100, cat. #GTX15323, GeneTex Inc.), and anti-Arginase I antibody (1:50, cat. #sc-271430, Santa Cruz Biotechnology). The antibody set-2 included anti-CD4 antibody (1:100, cat. #GTX44531, GeneTex Inc.) and anti-TNF-ζ antibody (1:75, cat. #sc-52746, Santa Cruz Biotechnology). The antibody set-3 was composed of anti-CD8 antibody (1:100, cat. #GTX16696, GeneTex Inc.) and anti-TNF-α antibody (1:75, cat. #sc-52746, Santa Cruz Biotechnology). After washing, the respective fluorescence-labeled secondary antibodies were applied and further incubated 1 h at room temperature. Nuclei were stained with DAPI. Finally, the sections were observed, photographed, and analyzed using Leica TCS SP5 II confocal microscope and LAS AF Lite 4.0 software.

### Results

### Preparation and Characterization of Mouse Anti-HSP90α Monoclonal Antibodies

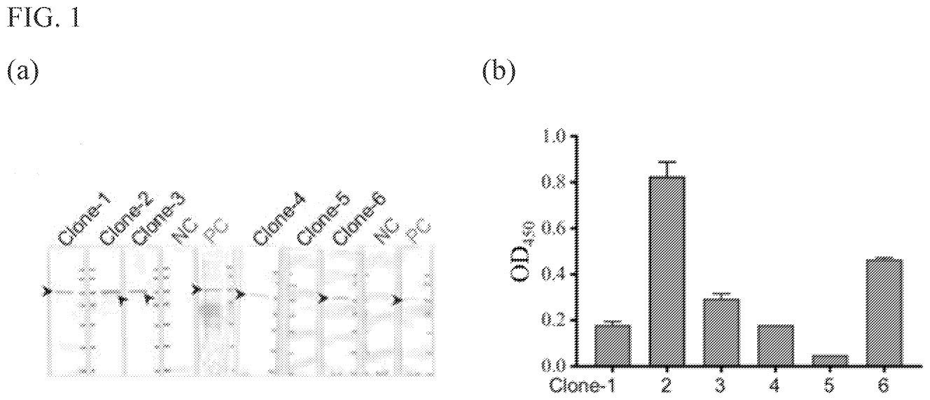

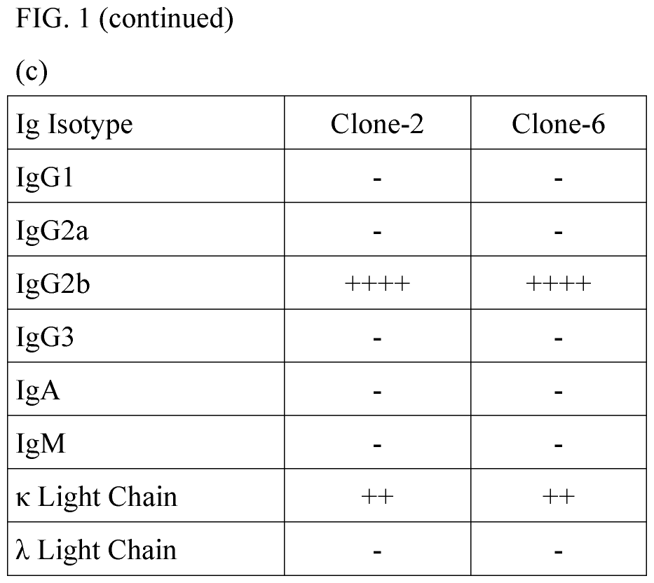

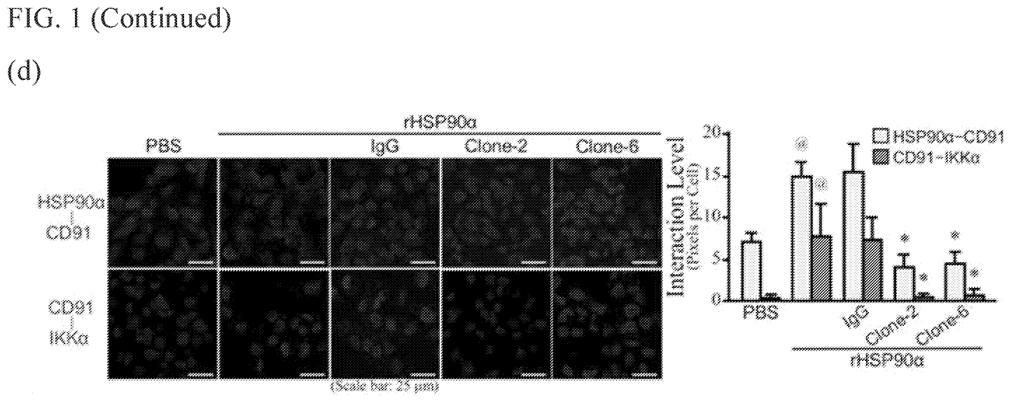

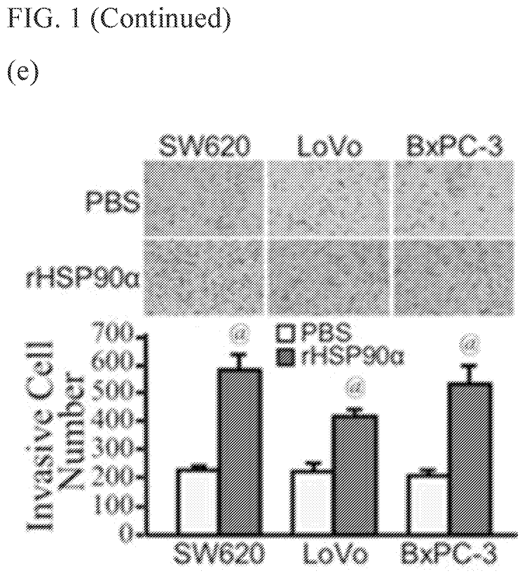

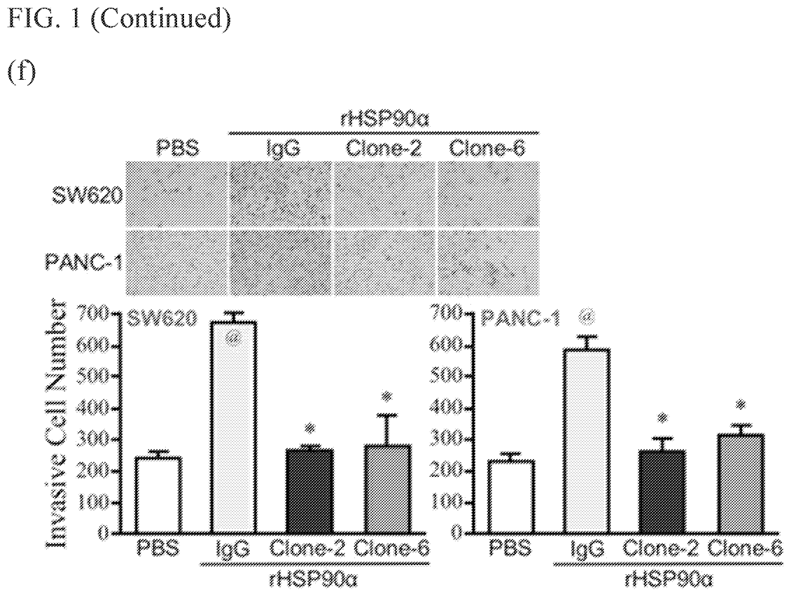

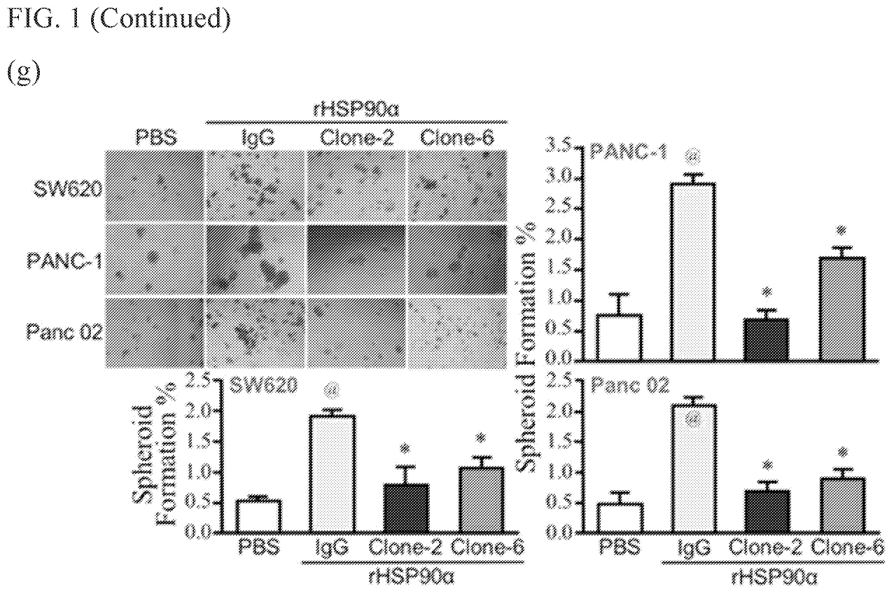

Six hybridoma clones were derived from the mice immunized with recombinant human HSP90α (rHSP90α). Their culture supernatants were collected for assaying the binding activities to HSP90α using standard western blotting and ELISA protocols. Antibodies produced by these hybridomas except clone-5 were shown to bind to the antigen rHSP90α (FIG. 1(a)). The binding activities were further quantified by ELISA, and Clone-2 and Clone-6 exhibited the top two HSP90α-binding levels and were thus chosen for next assays (FIG. 1(b)). The antibodies of the two clones were purified, and both were identified as IgG2b-isotype immunoglobulins containing κ light chains (FIG. 1(c)). They efficiently blocked the binding of eHSP90α with its cell-surface receptor CD91 and the downstream association of CD91 with IKKα (FIG. 1(d)). Given eHSP90α exerts its cancer-promoting effect through multiple mechanisms like inductions of cancer cell EMT, migration, invasion, and gain-of-stemness. Consistently with previous studies, our Transwell invasion assays revealed that rHSP90α acted as a potent inducer of cancer cell invasiveness (FIG. 1(e)), and the rHSP90α-induced cancer cell invasion could be drastically abolished by Clone-2 and Clone-6 anti-HSP90α antibodies (FIG. 1(f)). Additionally, rHSP90α-induced cancer cell gain-of-stemness, reflected by the increased spheroid-forming ability, could be also significantly inhibited by Clone-2 and Clone-6 antibodies (FIG. 1(g)). Taken together, our data suggested that anti-HSP90α monoclonal antibodies could be potentially developed and used to cope with eHSP90α-promoted malignancies.

### Humanization of Clone-2 Anti-HSP90α Antibody

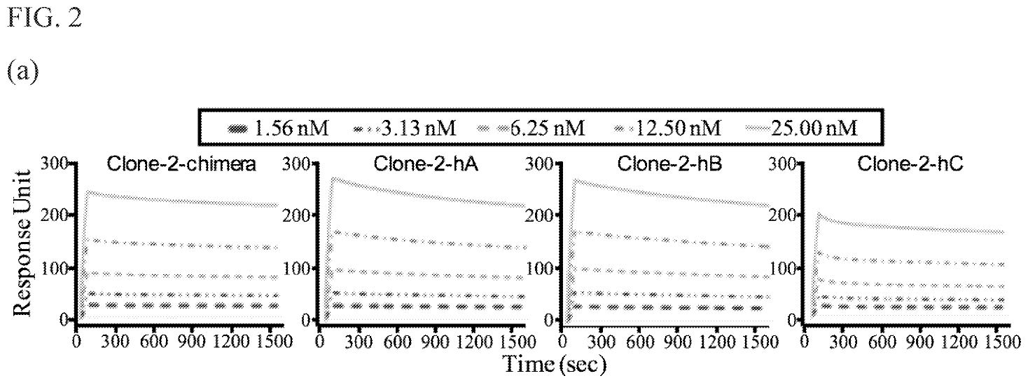

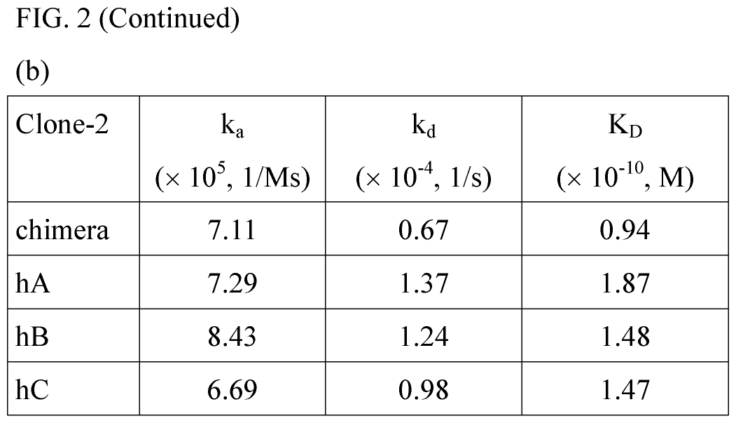

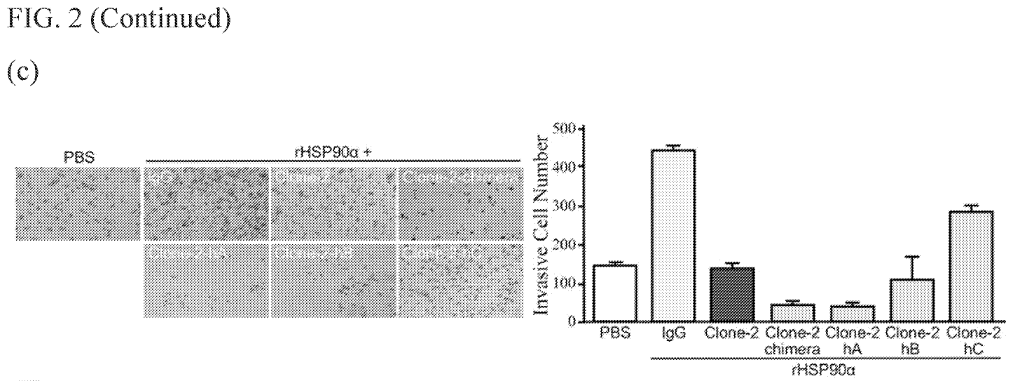

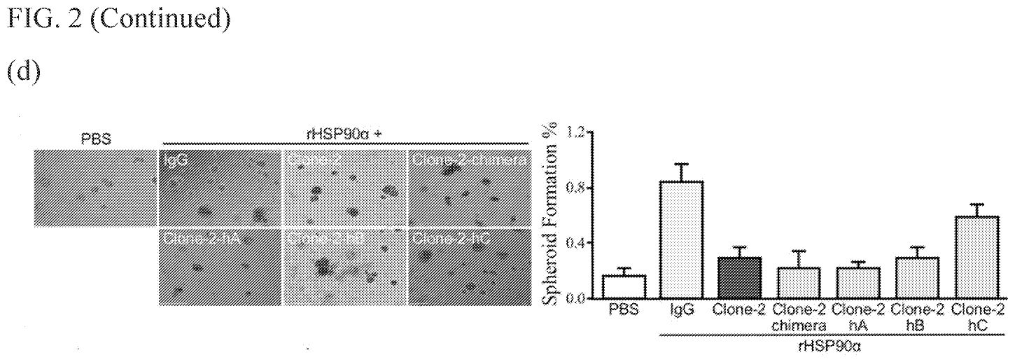

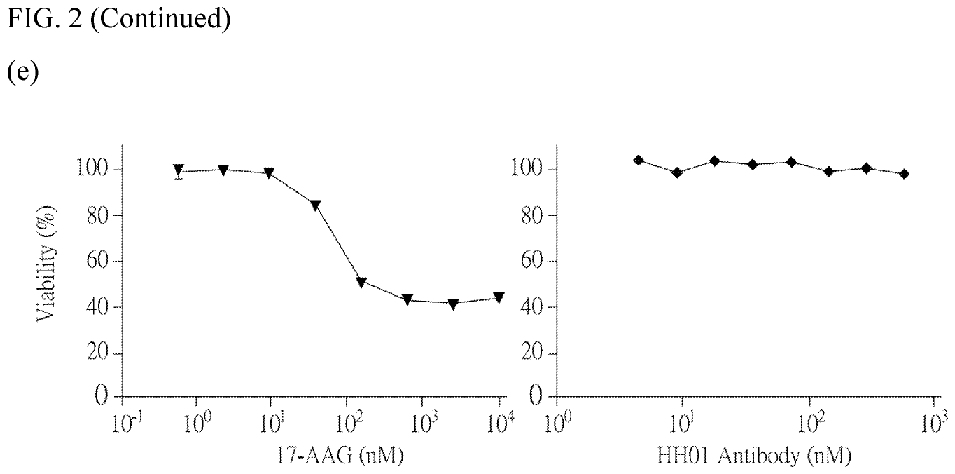

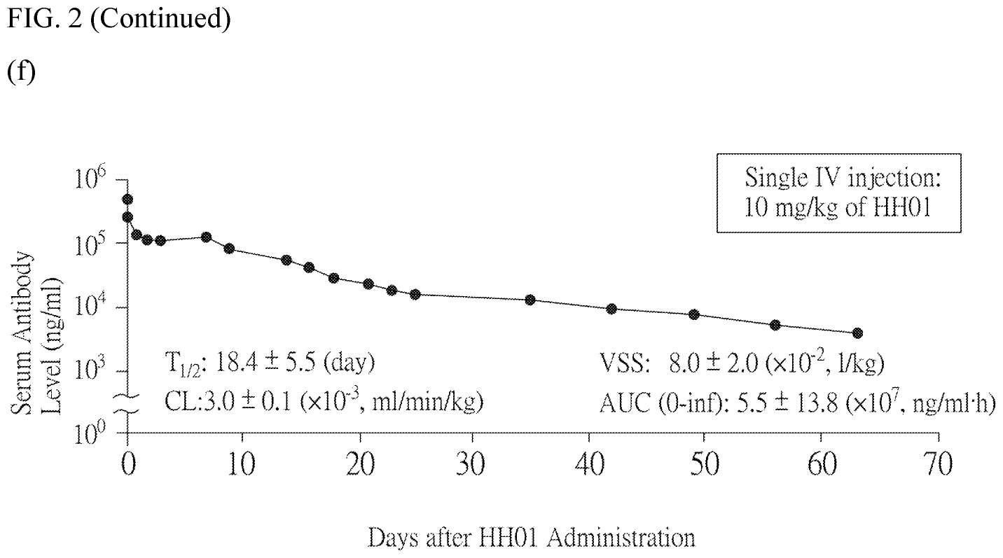

Next, we cloned and analyzed the cDNA sequences of the IgG variable domains of H and L chains (i.e. V_{H} and V_{L}) of Clone-2 and Clone-6 anti-HSP90α antibodies, respectively. The cDNA sequences and the encoding amino acid residues were listed in above Table 1 and Table 2. Clone-2 was further selected for humanization. The V_{H} and V_{L} DNA sequences were synthesized and inserted into the plasmid vectors just before the regions expressing human IgG constant domains of H and L chains (i.e. C_{H} and C_{L}), respectively, to express the recombinant IgG1 antibody called “Clone-2-chimera”. Moreover, we constructed 3 mutants of Clone-2-chimera, designated as Clone-2-hA, Clone-2-hB, and Clone-2-hC, intending to improve antibody properties in the regards of aggregation, protease cleavage, post-translational modifications, pharmacokinetics, and stability according to the suggestions of computer-assisted structural modeling and calculations. The mutation sites were listed in Table 3 below. These engineered humanized recombinant antibodies were expressed by ExpiCHO cells and were analyzed for HSP90α-binding affinities after purification. Kinetics assays were performed using Biacore T200 to determine the binding characteristics of Clone-2-chimera, Clone-2-hA, Clone-2-hB, and Clone-2-hC antibodies toward rHSP90α (FIG. 2(a)). The equilibrium dissociation constant (K_{D}) values of Clone-2-chimera, Clone-2-hA, Clone-2-hB, and Clone-2-hC antibodies were further calculated as 0.94×10⁻¹⁰, 1.87×10⁻¹⁰, 1.48×10⁻¹⁰, and 1.47×10⁻¹⁰ M, respectively (FIG. 2(b)), suggesting that the mutations in Clone-2-hA, Clone-2-hB, and Clone-2-hC antibodies did not significantly reduce the HSP90α-binding affinity of Clone-2-chimera.

TABLE 3

| | Mutation Sites |
|---|---|
| Clone-2-hA | Light chain: L3V, K45Q, S74T, R77S, G81E |
| | Heavy chain: V4L, K15G, K19R, K43Q, Q75K, |
| | F76N, M77T, K83R, M89V |
| Clone-2-hB | Light chain: L3V, V27L, K45Q, S74T, R77S, G81E |
| | Heavy chain: V4L, K15G, K19R, K43Q, Q75K, |
| | F76N, M77T, N82S. K83R, M89V |
| Clone-2-hC | Light chain: L3V, V27L, K45Q, S74T, R77S, G81E |
| | Heavy chain: V4L, K15G, K19R, K43Q, K52L, |
| | Y55S, Q75K, F76N, M77T, N82S. K83R, M89V |

To preliminarily evaluate the anti-cancer efficacies of these humanized anti-HSP90α antibodies, we assayed their inhibitory activities on rHSP90α-induced PDAC cell invasion and spheroid formation. The results of the Transwell invasion assay showed that Clone-2-chimera and Clone-2-hA were superior to Clone-2-hB, Clone-2-hC, and their parental Clone-2 in suppressing rHSP90α-induced invasiveness of human PDAC PANC-1 cells (FIG. 2(c)). In the spheroid-forming assay, Clone-2-hA still showed a most outstanding efficacy in suppressing rHSP90α-induced spheroid formation of PANC-1cells (FIG. 2(d)). Therefore, Clone-2-hA was finally chosen and named as HH01 antibody for further in vitro and in vivo assays. The solubility of HH01 antibody in PBS was >10 mg/ml and the Dynamic Light Scattering assay revealed that HH01 antibody had a size of average diameter 19.33 nm from a range of 10-50 nm (not shown in the figure), suggesting that HH01 antibody did not easily form aggregate. Considering clinical use of small-molecule HSP90 inhibitors including geldanamycin and resorcinol derivatives caused retinal photoreceptor cell damage when they accumulated at retina to some levels, we further evaluated whether HH01 antibody exhibited any toxicity in non-cancerous retinal cells by investigating the effect of HH01 antibody on the viability of ARPE-19 retinal pigmented epithelial cells. As shown in FIG. 2(e), 17-AAG exhibited a cytotoxic profile to ARPE-19 cells with a ICso of 64.7 nM, however, HH01 antibody did not cause any obvious cytotoxicity in ARPE-19 cells even when its concentration reached at 594.3 nM (100 μg/ml). Additionally, pharmacokinetics of HH01 antibody was studied in male mice, which showed that HH01 antibody had a half-life time (T_{1/2}) of 18.4 days with a clearance rate at 3×10⁻³ ml/min/kg and a volume in steady state of 80 ml/kg in mouse blood (FIG. 2(f)). The results showing the long terminal T_{1/2} and high serum exposure level and after intravenous dosing revealed that HH01 antibody was suitable for further development.

### Identification of the Epitopes of HH01 Antibody

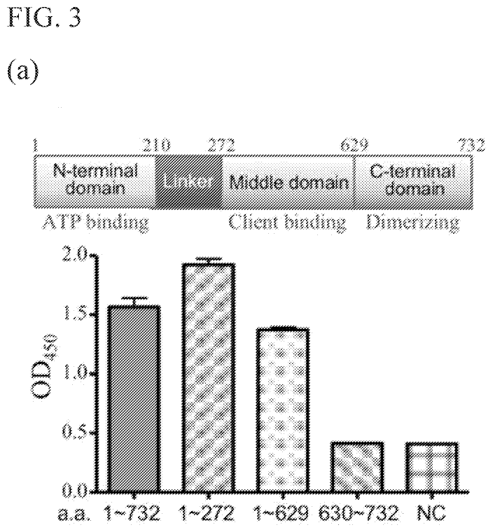

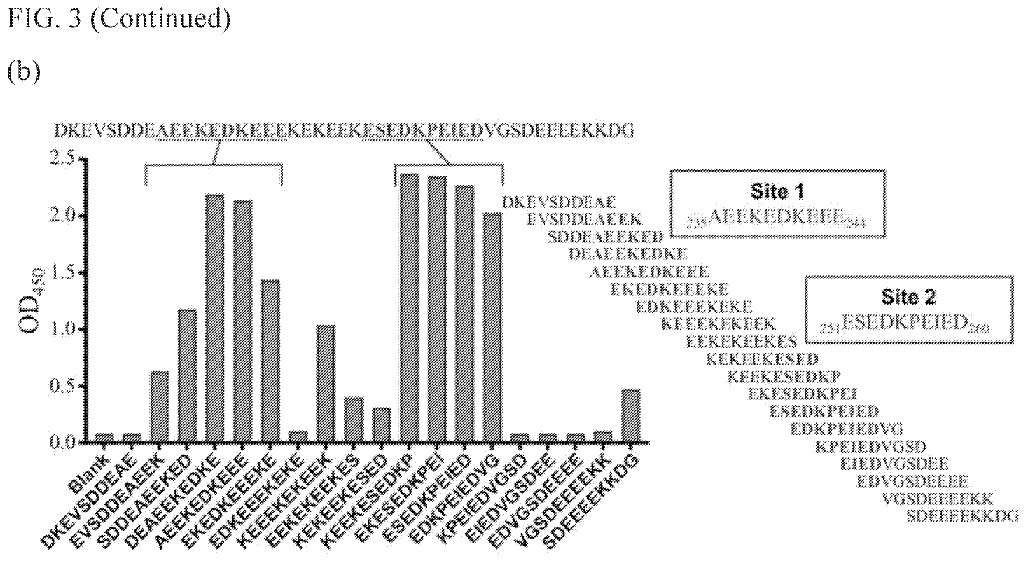

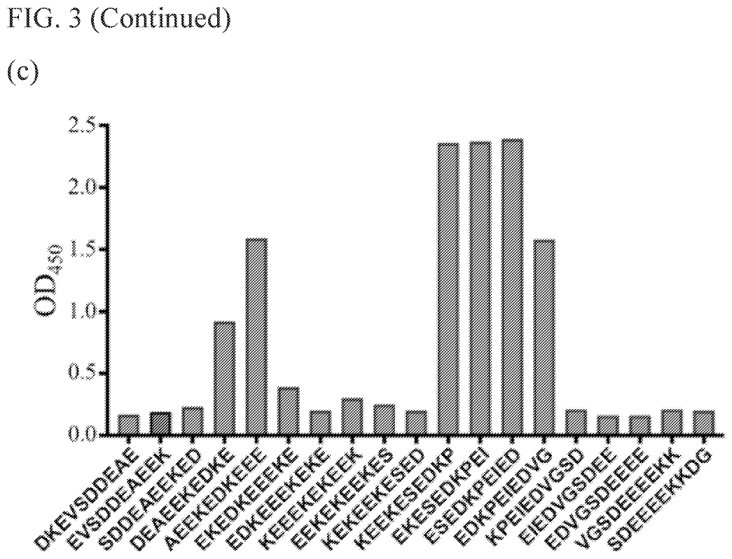

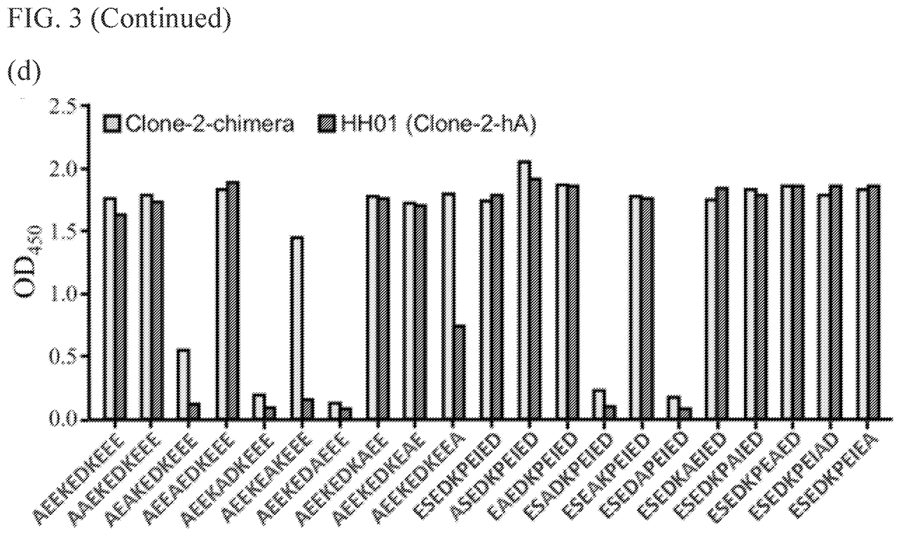

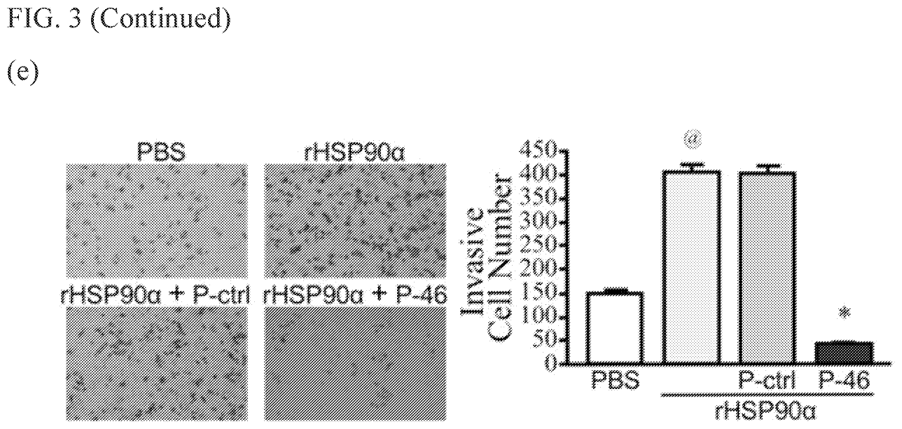

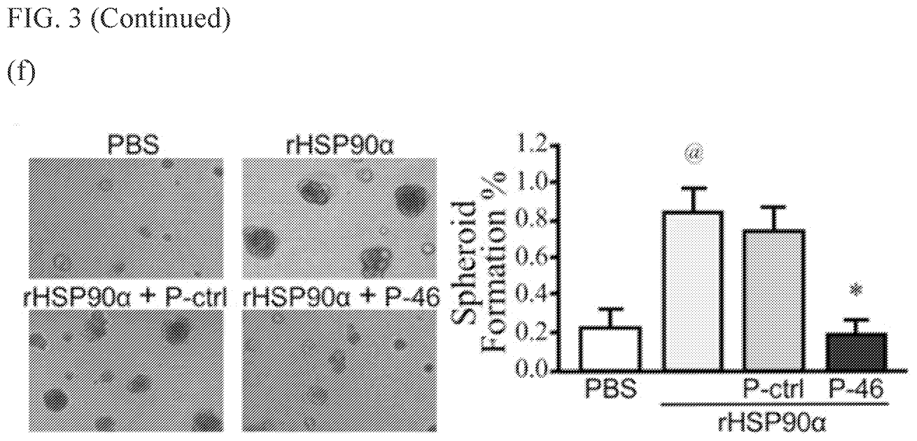

As listed in the above Table 3, HH01 antibody is capable of binding specifically to the human HSP90α with novel amino acid (a.a.) sequences in the complementarity determining regions (CDRs), and therefore we intended to identify which region of HSP90α was bound by HH01 antibody. In a preliminary study, we employed the strategy of HSP90α domain scanning to delineate the HSP90α region responsible for the binding with Clone-2 antibody. The full-length HSP90α (a.a. 1-732) and its 3 truncated fragments including the N-terminal plus Linker domains (a.a. 1-272), N terminus through Middle domain (a.a. 1-629), and C-terminal domain (a.a. 630-732) were cloned, expressed, and purified. Subsequently, the binding activities of Clone-2 antibody to the full-length and truncated HSP90α were assayed by a conventional ELISA method. The results showed that Clone-2 antibody bound to the a.a. 1-272 region as well as the full-length protein (FIG. 3(a)), suggesting that the binding epitope(s) of Clone-2 antibody could be within the N-terminal and Linker domains (a.a. 1-272) of HSP90α. Furthermore, the strategy of peptide scanning was adopted to delineate the binding epitope(s) of Clone-2-chimera and HH01 antibodies. A peptide library was established to consist of a series of 10-a.a. peptides with an 8-a.a. overlap between any two consecutive peptides to scan the sequence of the a.a. 21-272 region of HSP9060 . The binding activities of Clone-2-chimera and HH01 antibodies to this series of 10-a.a. peptides were assayed by ELISA. The results revealed that the probable epitope sites of Clone-2-chimera antibody were ₂₃₅AEEKEDKEEE₂₄₄ and ₂₅₁ESEDKPEIED₂₆₀ of HSP90α (FIG. 3(b)). Consistently, these two sites could also serve as the binding epitopes of HH01 antibody (FIG. 3(c)). Based on the sequences of these two sites, we synthesized two series of 10-a.a. peptides with a.a. residues sequentially substituted with alanine. The strategy of alanine scanning was employed to investigate the a.a. residues critical for the binding of Clone-2-chimera and HH01 antibodies to the epitope sites. The results showed that the binding activities of Clone-2-chimera antibody to the epitope sites were decreased drastically when E237, E239, K241, E253, and K255 were replaced with alanine (FIG. 3(d)). Besides, one more D240 was also responsible for the binding of HH01 antibody (FIG. 3(d)). According to the above epitope mapping results, we synthesized a peptide with a sequence of the a.a. 227-272 region of HSP90α. This peptide competitively suppressed rHSP90α-induced PANC-1 cell invasion (FIG. 3(e)) and spheroid formation (FIG. 3(f)), confirming that the HSP90α region containing the epitope sites could be a target of novel anti-cancer strategies.

### Evaluation of the Prophylactic and Therapeutic Efficacies of HH01 Antibody in Desmoplastic Mouse PDAC Transplant Model

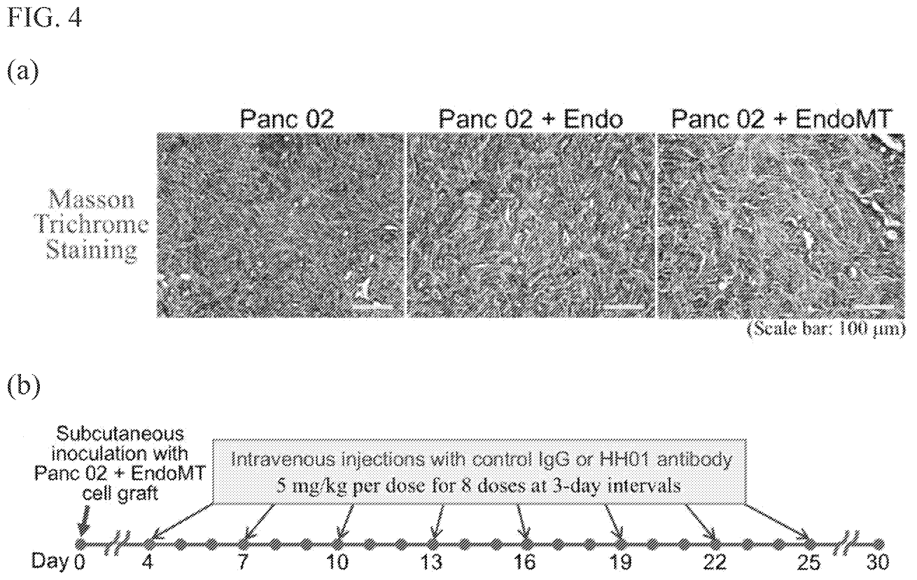

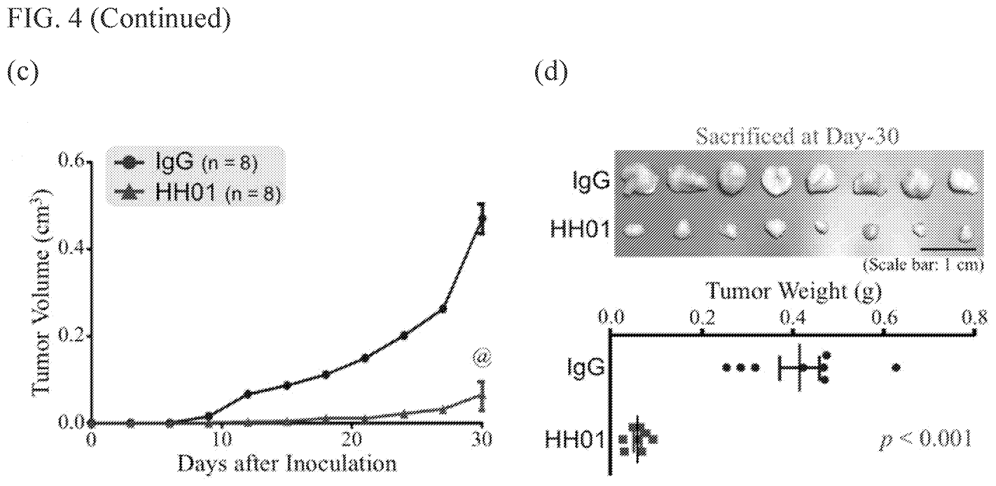

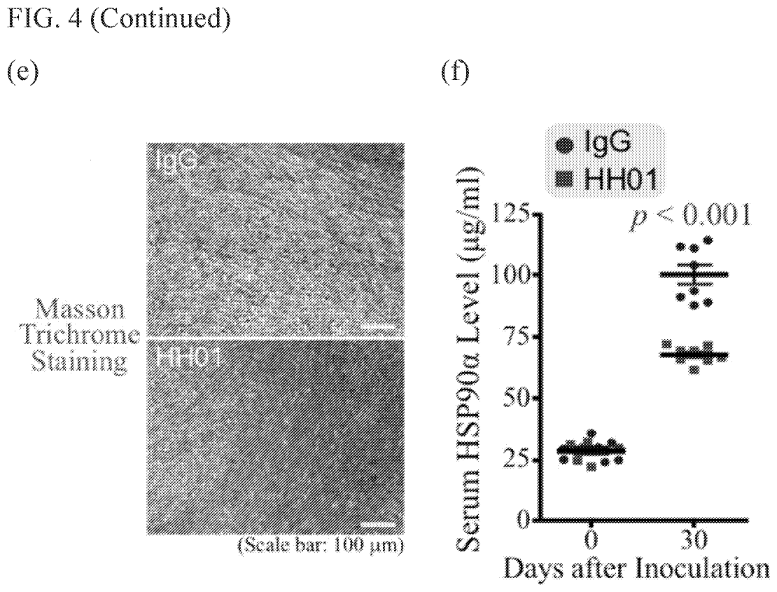

HH01 antibody was for further in vivo anti-cancer assays. In our previous studies, EndoMT-derived CAFs significantly facilitated the tumor growth of pancreatic adenocarcinoma Panc 02 cell grafts. Our result of Masson trichrome staining further revealed that the EndoMT cells-involved Panc 02 tumor exhibited a significant level of desmoplasia which was not observed in the tumor derived from Panc 02 cells alone (FIG. 4(a)). To investigate the in vivo anti-cancer activity of HH01 antibody, we followed our previous schedule using mouse antibody and assayed its cancer-preventive activity in EndoMT-involved mouse PDAC model. C57BL/6 mice were subcutaneously inoculated with Panc 02 cells plus EndoMT cells. On post-inoculation Day-4, the mice were further intravenously administered with 5 mg/kg per dose of control IgG or HH01 antibody for 8 doses at 3-day intervals (FIG. 4(b)). As shown in FIG. 4(c), the kinetics of the tumor growth of EndoMT-involved Panc 02 cell grafts was potently suppressed by HH01 antibody. All mice were sacrificed on post-inoculation Day-30 and their tumors were taken for weighing. As expected, HH01 treatment significantly repressed the tumor sizes of Panc 02 plus EndoMT cell grafts (p <0.001, FIG. 4(d)). The tumor desmoplasia was also significantly prevented by HH01 treatment (FIG. 4(e)). Additionally, increased eHSP90α levels were detected in the sera of our desmoplastic mouse PDAC model, and the serum HSP90α levels were significantly reduced in the mice upon HH01 treatment (FIG. 4(f)). These data suggest that HH01 antibody is a potent agent to prevent EndoMT-promoted tumor growth of PDAC cells.

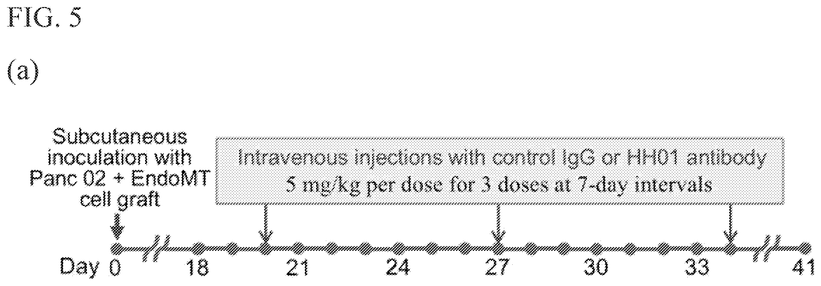

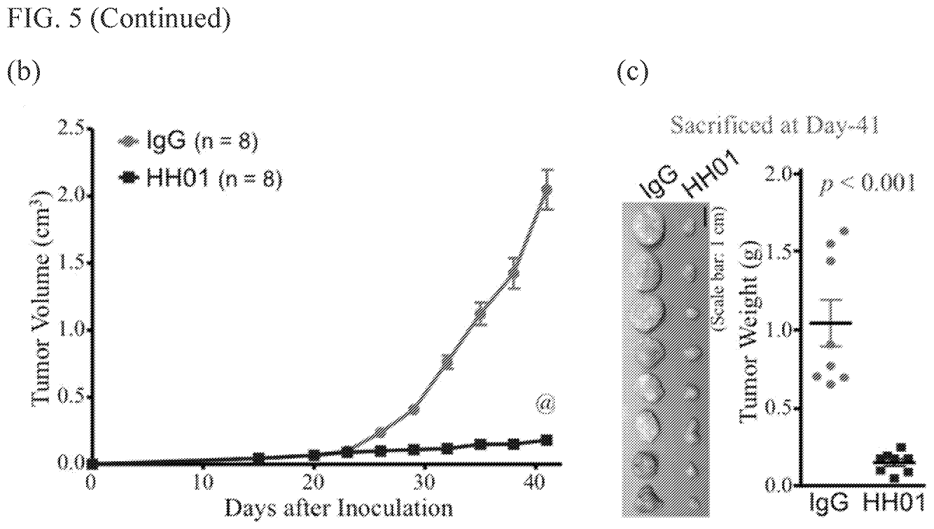

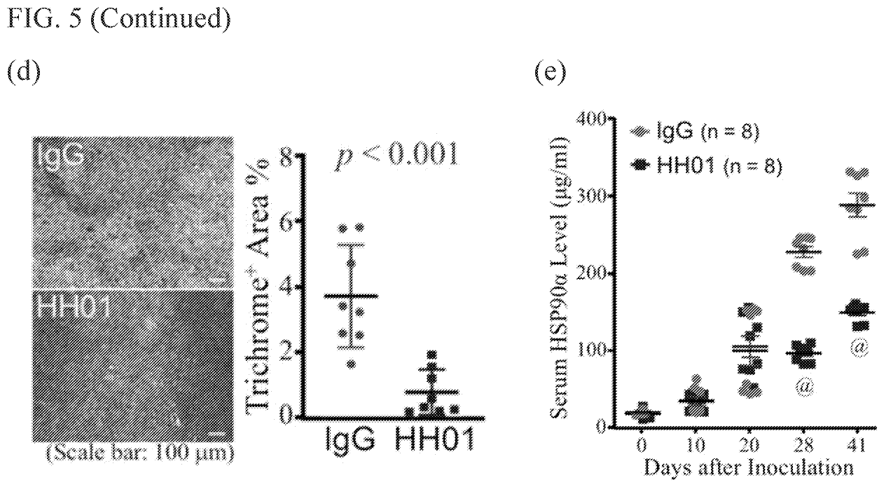

To evaluate the therapeutic efficacy of HH01 antibody in desmoplastic mouse PDAC model, C57BL/6 mice were subcutaneously inoculated with Panc 02 plus EndoMT cell grafts. On post-inoculation Day-20, the mice bore tumors with an average size of 75-mm³ and started to be intravenously injected with 5 mg/kg per dose of control IgG or HH01 antibody for 3 times at 7-day intervals (FIG. 5(a)). The result showed that HH01 treatment totally suppressed the tumor growth kinetics of Panc 02 plus EndoMT cell grafts (FIG. 5(b)). The tumors were taken on Day-41 for weighing and the data revealed that the tumor growth was indeed repressed in HH01-treated mouse group (FIG. 5(c)). The HH01 treatment also resulted in significant decreases of tumor desmoplasia (FIG. 5(d)) and serum HSP90α levels (FIG. 5(e)). Taken together, these data suggest that HH01 antibody can be developed as a therapeutic agent to effectively treat desmoplastic PDAC.

### Evaluation of the Therapeutic Efficacy of HH01 Antibody in Desmoplastic Humanized Mouse PDAC Transplant Model

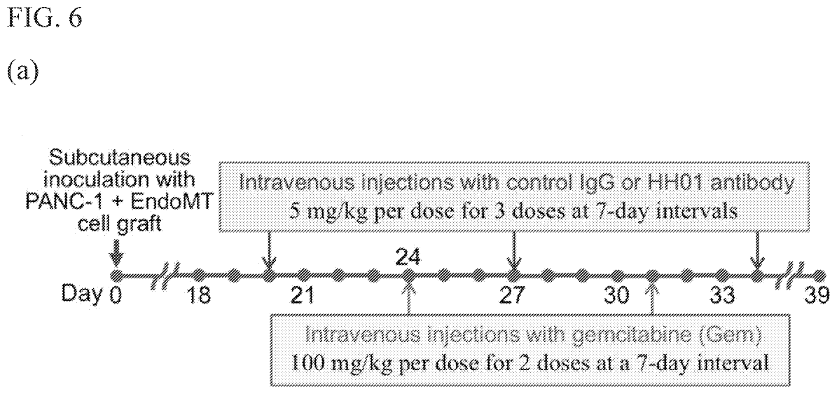

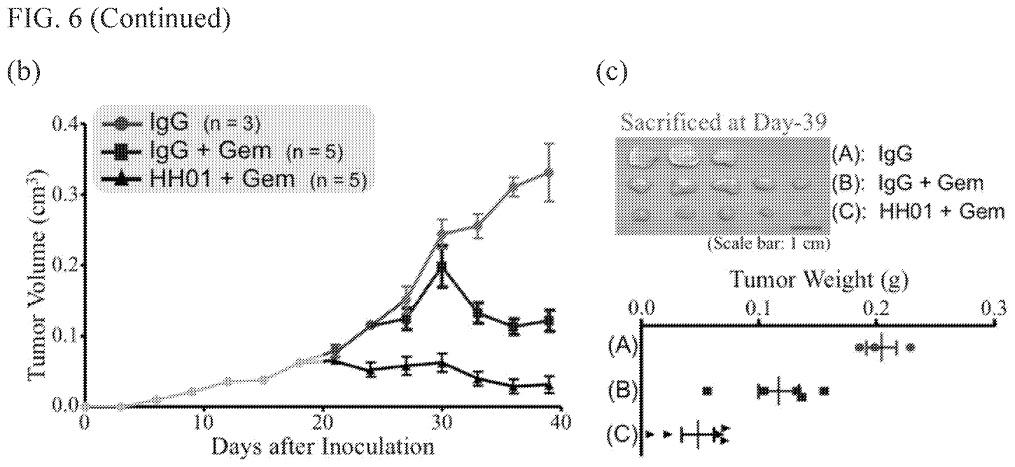

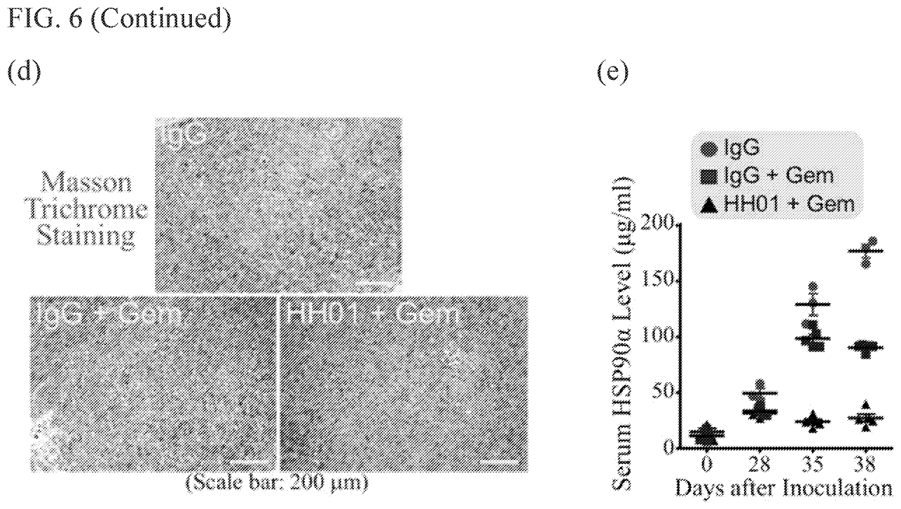

We also evaluated the therapeutic efficacy of HH01 antibody in desmoplastic humanized mouse PDAC model. hHSC-transplanted ASID mice were subcutaneously inoculated with a mix of human pancreatic adenocarcinoma PANC-1 cells with EndoMT-derived cells. On post-inoculation Day-20, the mice were divided into 3 groups for further treatments (FIG. 6(a)). The Group A mice (n=3) were intravenously injected with 5 mg/kg per dose of control IgG for 3 doses at 7-day intervals. The Group B mice (n=5) were injected trice with control IgG plus twice with gemcitabine in comparison with the Group C mice (n=5) injected trice with HH01 antibody plus twice with gemcitabine. The mice received the first 5 mg/kg of IgG (Group B) or HH01 (Group C) on Day-20 after inoculation, and 4 days later, they were further intravenously injected with the first 100 mg/kg of gemcitabine. The other 2 doses of control IgG or HH01 antibody and another dose of gemcitabine were administered at respective 7-day intervals (FIG. 6(a)). Developing tumors were superficially measured every 3 days until post-inoculation Day-39, and the tumor growth curves were plotted as FIG. 6(b). Compared with the mice treated with IgG alone, the mice treated with IgG plus gemcitabine had significantly repressed tumors after the second administration of gemcitabine. However, in the mice treated with HH01 plus gemcitabine, a more effective repression of the tumor growth was observed only 4 days after the first-dose HH01 injection. The tumors further shrank after the second-dose gemcitabine and the third-dose HH01 were administered (FIG. 6(b)). All mice were sacrificed on Day-39, and the tumors from the mice treated with HH01 plus gemcitabine were significantly repressed (FIG. 6(c)). The combination of HH01 antibody with gemcitabine also exhibited potent efficacies in suppressing tumor desmoplasia (FIG. 6(d)) and serum HSP90α levels (FIG. 6(e)). These data confirmed that HH01 antibody could be an effective therapeutic agent to target desmoplastic PDAC.

### Evaluation of the Therapeutic Efficacy of HH01 Antibody in K-Ras^{G12D}-Induced Desmoplastic Mouse PDAC Model

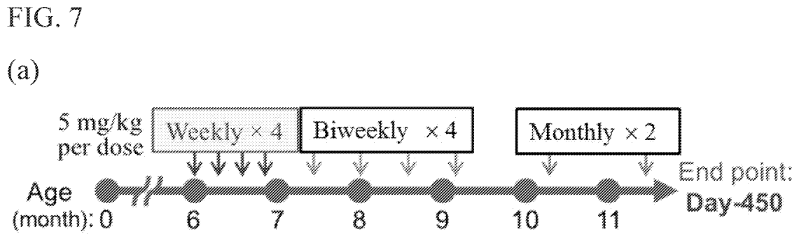

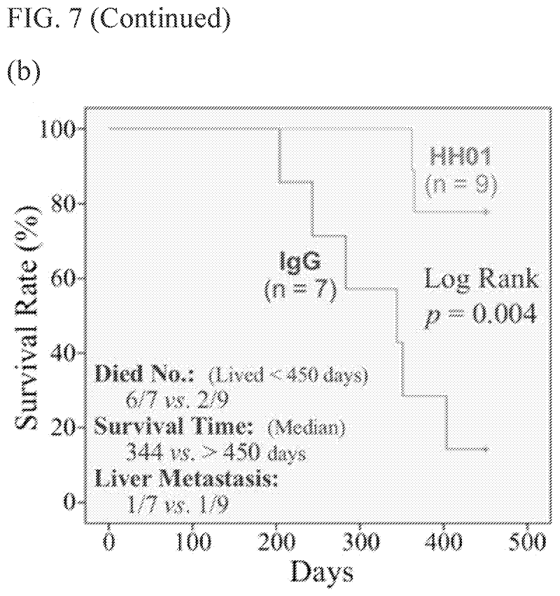

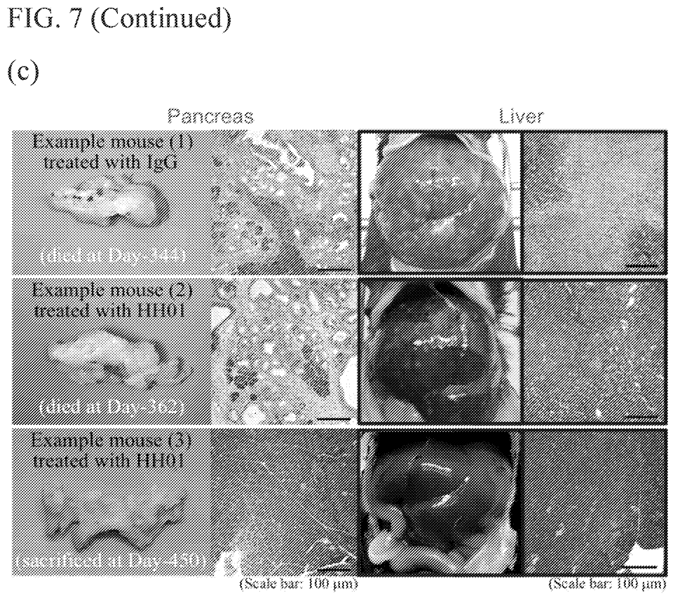

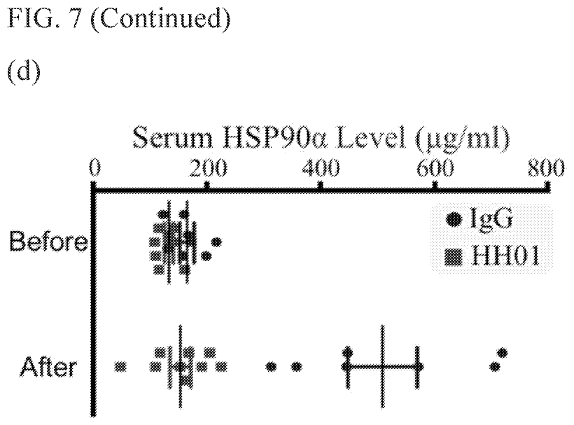

Next, we evaluated the therapeutic efficacy of HH01 antibody in spontaneously PDAC-developing mouse model. Clinically, >90% of PDAC patients harbor activated K-Ras mutants in their adenocarcinoma cells. In transgenic mouse models, a knock-in expression of activated mutant K-Ras^{G12D} in pancreatic ductal cells causes a PDAC development through a process of acinar-to-ductal metaplasia (ADM), pancreatic intraepithelial neoplasia (PanIN), and the resultant PDAC. These pancreatic K-Ras^{G12D}-expressing (LSL-KrasG12D/Pdx1-Cre) mice develop ADM as early as 3 months after birth and further develop PDAC lesions at 6 months of age. The results of Masson trichrome staining revealed that a little of small-sized desmoplastic regions could be detected from the pancreatic tissues of 3-month-old LSL-KrasG12D/Pdx1-Cre mice, and furthermore desmoplasia was extensively detected from the pancreatic tissues of 6-month and older LSL-KrasG12D/Pdx1-Cre mice but not LSL-KrasG12D control mice. To evaluate the therapeutic efficacy of HH01 antibody, LSL-KrasG12D/Pdx1-Cre mice did not receive any treatment until they had developed PDAC at 6 months of age. They were intravenously injected with 5 mg/kg per dose of control IgG or HH01 antibody in a totally 10-dose schedule: 4 doses administered weekly, followed by 4 doses administered biweekly, and finally 2 doses administered monthly (FIG. 7(a)). The whole experimental was set an end point on Day-450 and the survival curves of IgG or HH01-treated mice were plotted by Kaplan-Meier method (FIG. 7(b)). While 6 out of 7 IgG-treated mice died <450 days, 7 of 9 HH01-treated mice still survived on Day-450. The median survival times of IgG-and HH01-treated mice were 344 days and >450 days, respectively (p=0.004 by Log rank analysis). Differing from the smooth pancreatic morphology of normal C57BL/6 mice or LSL-KrasG12D control mice, all LSL-KrasG12D/Pdx1-Cre mice had nodular pancreases whatever the mice had received HH01 therapy or not (FIG. 7(c)). However, Masson trichrome staining revealed that pancreatic desmoplasia was drastically recovered in those mice treated with HH01 and still surviving on Day-450 (FIG. 7(c)). One IgG-treated and one HH01-treated mice were observed to have tumor nodules on the appearance of livers, but nevertheless the HH01-treated mouse obviously had fewer nodules than IgG-treated mouse (FIG. 7(c)). Masson trichrome staining also revealed that HH01-treated mouse exhibited a significant improvement of liver desmoplasia (FIG. 7(c)). Additionally, mouse serum samples were collected monthly from 1 day before the start of IgG or HH01 treatments. The HSP90α levels of the last serum samples collected near mouse expiries were compared with those of samples collected before IgG or HH01 treatments (i.e. after vs. before treatments). Our data showed that elevation of serum HSP90α levels in LSL-KrasG12D/Pdx1-Cre mice could be effectively abolished by HH01 therapy (FIG. 7(d)).

### Evaluation of the Therapeutic Efficacy of HH01 Antibody in M2-Macrophage-Exacerbated Mouse PDAC Model

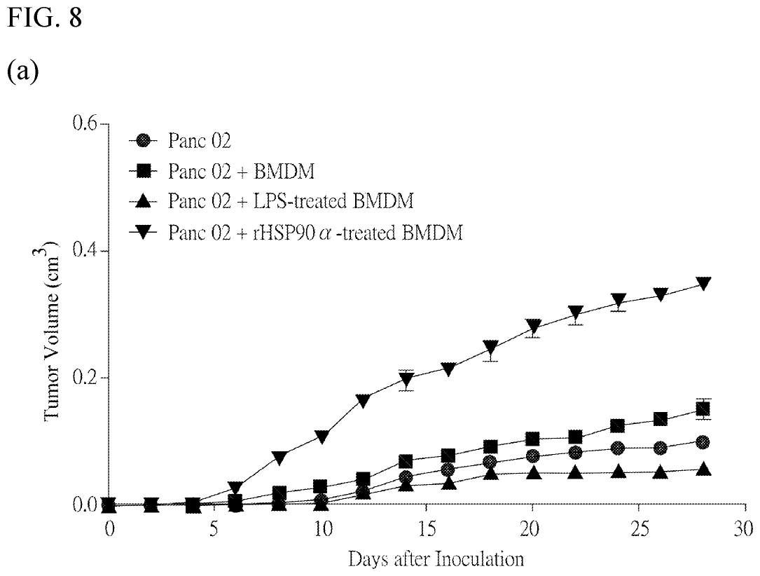

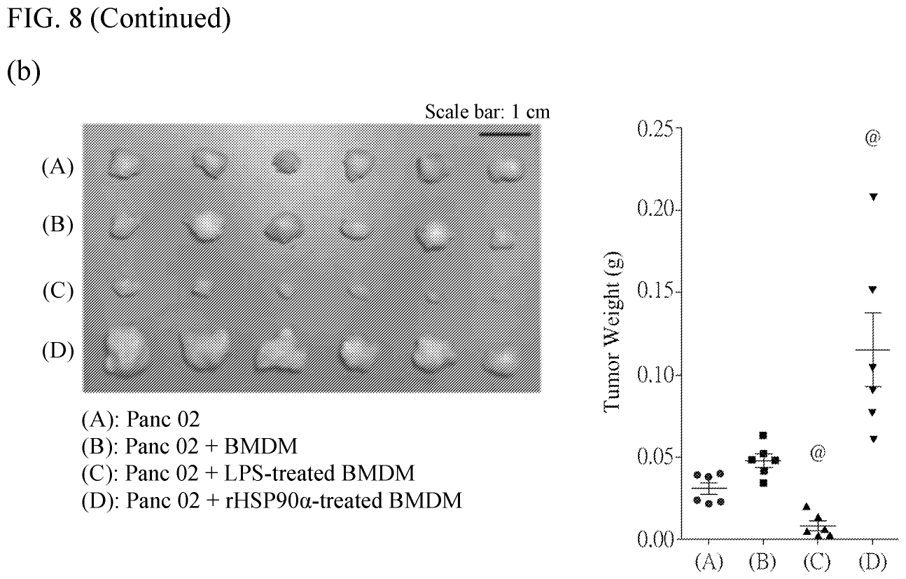

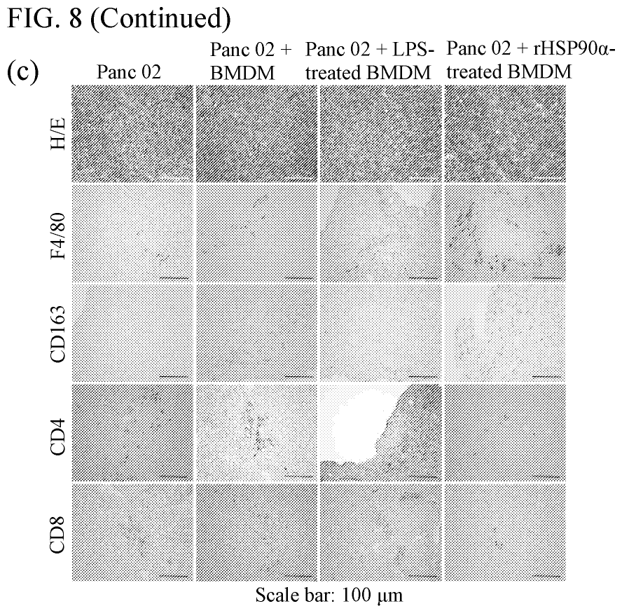

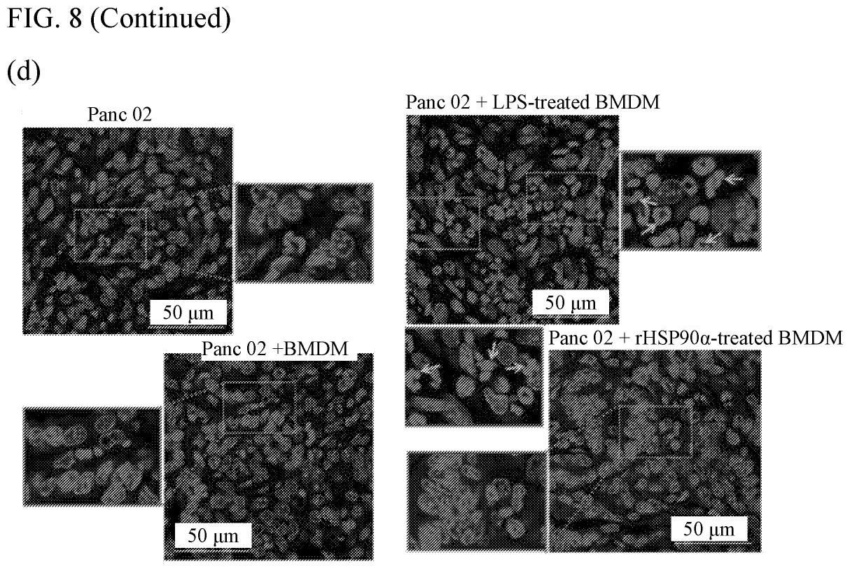

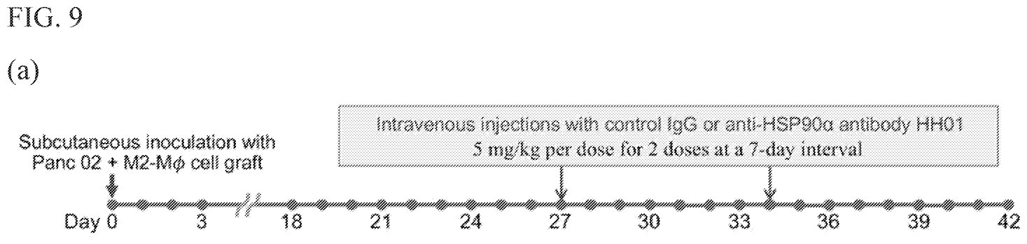

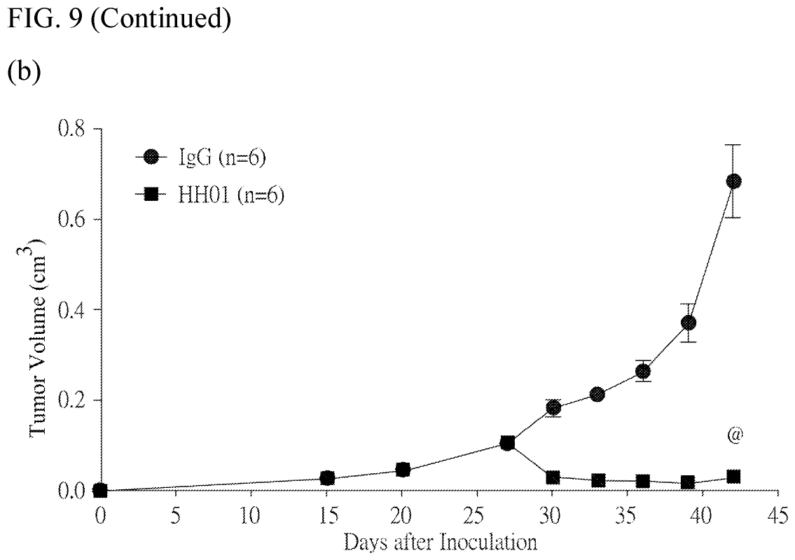

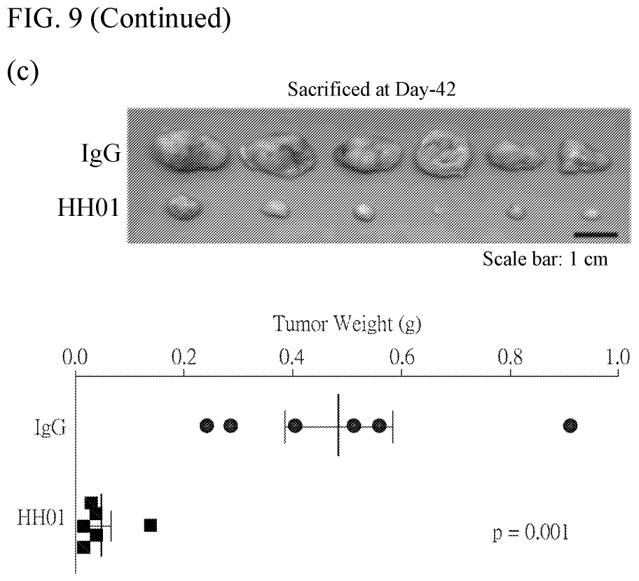

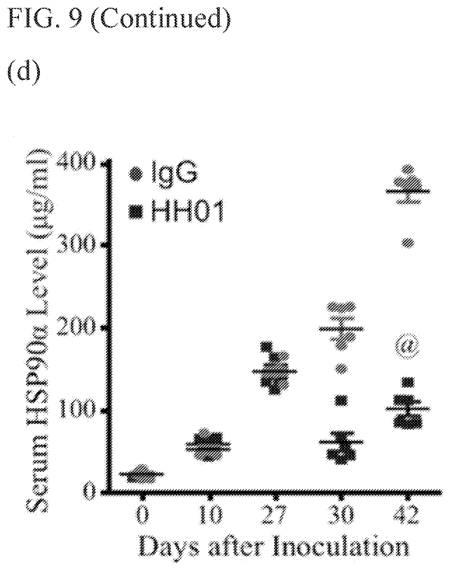

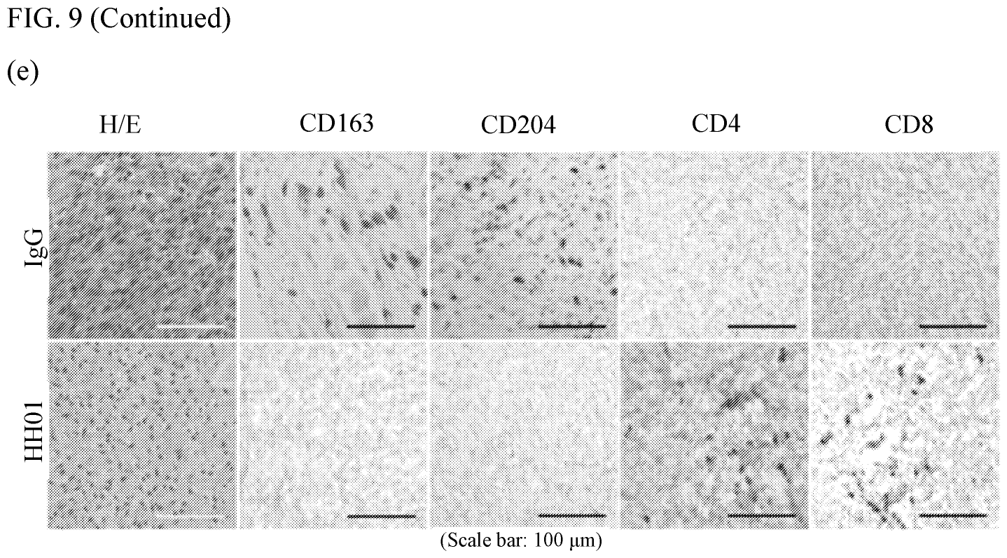

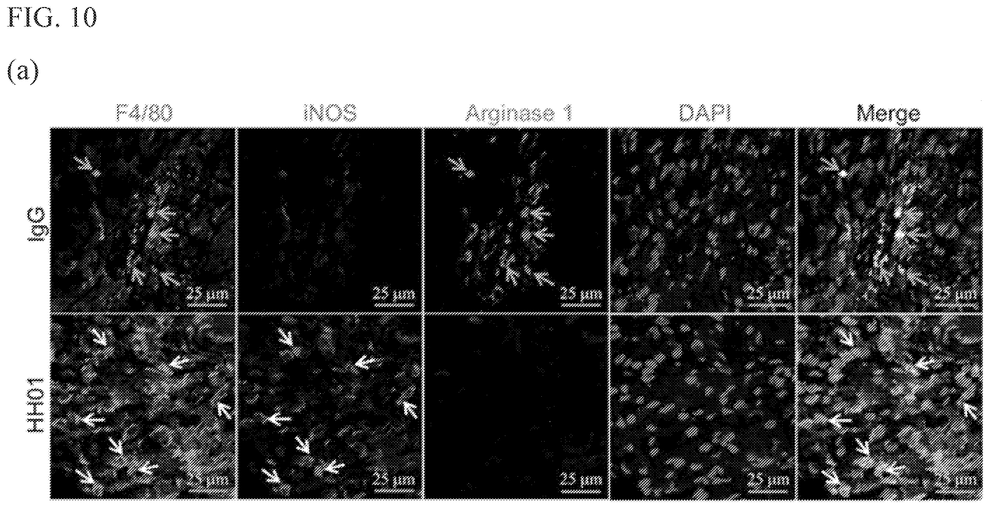

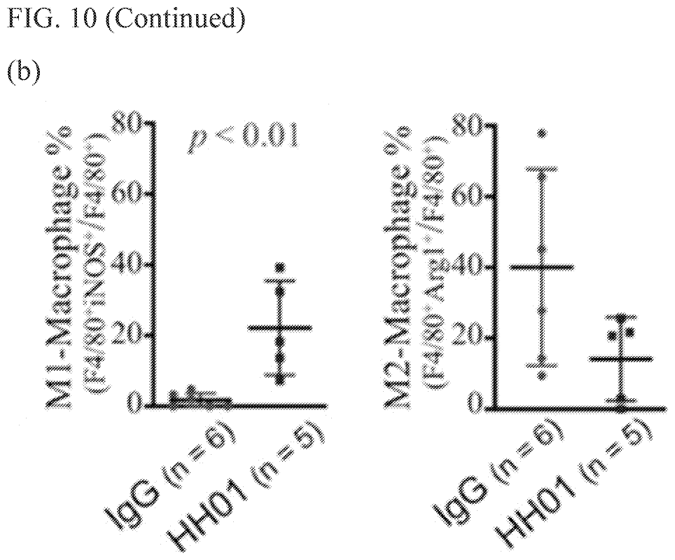

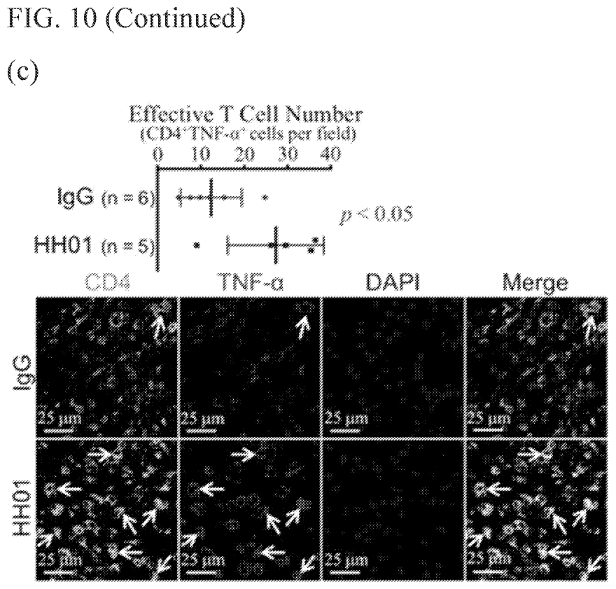

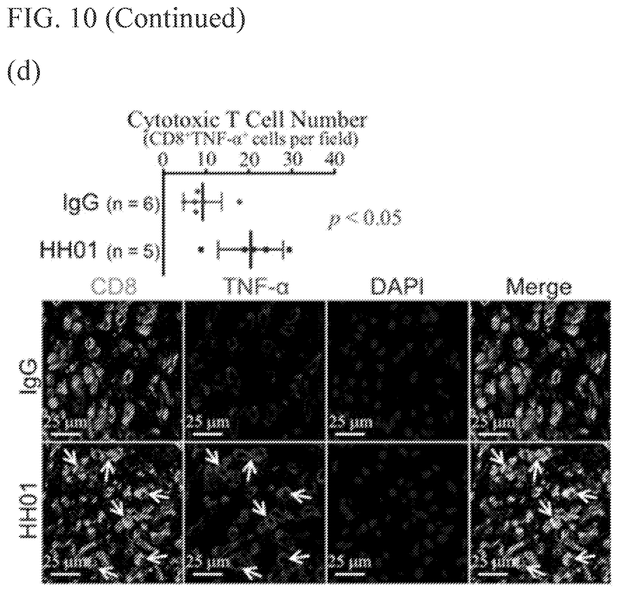

To establish an M2-macrophage-involved mouse PDAC model, we mixed rHSP90α-treated BMDMs with Panc 02 cells for subcutaneous inoculation into C57BL/6 mice. For comparison with M1-macrophages, Panc 02 cells plus LPS-treated BMDMs were inoculated into another group of mice. Growth of the “Panc 02 plus rHSP90α-treated BMDM” grafts started from Day-6 after inoculation and the tumor growth continued faster than other groups of grafts (FIG. 8(a)). On Day-30 after inoculation, all mice were sacrificed and their tumors were taken for weighing. As shown in FIG. 8(b), rHSP90α-treated BMDMs significantly promoted Panc 02 tumor growth, which was in contrast to the tumor-suppressive effect resulted from LPS-treated BMDMs. The results of immunohistochemical analyses revealed that the tumor tissues derived from the “Panc 02 plus rHSP90α-treated BMDM” grafts contained more CD163⁺ cells but significantly reduced levels of CD4⁺ and CD8⁺ cells when compared with the tumor masses taken from other groups of mice (FIG. 8(c)). In the mouse group with LPS-treated BMDMs, the repressed tumor tissues contained relatively higher levels of CD4⁺ and CD8⁺ cells. Nuclear staining with 4′,6′-diamidino-2-phenylindole (DAPI) also showed elevated levels of chromatin condensation and nuclear fragmentation (FIG. 8(d)), suggesting that LPS-induced M1-macrophages recruited CD4⁺ and CD8⁺ immune cells and caused more cell apoptosis to suppress Panc 02 tumor growth. Therefore, these results conclude that eHSP90α-induced M2-type macrophages exhibit potent immunosuppressive and tumor-promoting activities. Furthermore, we evaluated the therapeutic efficacy of HH01 antibody in this M2-macrophage-promoted PDAC model. C57BL/6 mice were subcutaneously inoculated with Panc 02 plus rHSP90α-treated BMDM cell grafts. On Day-27 post-inoculation, each mouse bore an approximately 0.1-cm³ tumor and started to be treated with 5 mg/kg of control IgG or HH01 antibody (FIG. 9(a)). Considering the half-life of HH01 in mouse blood >18.4 days, the action of HH01 antibody was boosted by the second dose after a 7-day interval. As shown in FIG. 9(b), the drastic tumor shrinkage upon HH01 treatment was observed immediately after 3 days. All mice were sacrificed on Day-42, and the result showed that the tumors shrank significantly in HH01-treated mice when compared with those continuously growing tumors in control mouse group (p=0.001, FIG. 9(c)). Consistently, the elevation of serum HSP90α levels was also significantly suppressed after HH01 therapy (FIG. 9(d)). The immunohistochemical analyses of the tumor samples also revealed that M2-macrophage-suppressed CD4⁺ and CD8⁺ cell levels were effectively restored in accordance with the M2-macrophage reduction upon HH01 therapy (FIG. 9(e)). These results were confirmed by the immunohistofluorescent staining results. The increase in F4/80⁺iNOS⁺ (M1) cells and decrease in F4/80⁺Arginasel⁺ (M2) cells were observed from HH01-treated mouse tissues (FIGS. 10(a) and 10(b)). Consistently, the levels of CD4⁺TNF-α⁺ (effective T) cells and CD8⁺TNF-α⁺(cytotoxic T) cells were both increased in response to the therapy with HH01 antibody (FIGS. 10(c) and 10(d)). These data suggest that HH01 antibody is an efficacious agent to suppress M2-macrophage-exacerbated PDAC and improve the immunity of tumor microenvironment.

### Conclusion

Desmoplasia is a hallmark of many malignancies, which is tightly associated with rapid tumor growth, metastatic occurrence, and refractory therapeutic outcome. Agents and strategies targeting desmoplasia to alleviate malignant progression and improve therapeutic efficacy are still the unmet medical needs for the cancer patients. A big production of cancer-associated fibroblasts (CAFs) is one of main causes of desmoplasia, and endothelial-mesenchymal transition (EndoMT) of endothelial cells provides a rich source for CAFs. EndoMT-derived CAFs recruit a large amount of myeloid-derived macrophages into tumor, and facilitate polarization of these macrophages toward M2 type. The M2-type macrophages do not only secrete IL-10 and TGF-β for suppressing effective and cytotoxic T cells, but also produce VEGF, bFGF, and PDGF to stimulate tumor angiogenesis. Additionally, these M2-type macrophages express and secrete a great amount of eHSP90α and create an eHSP90α-rich tumor microenvironment which has the advantage of cancer cell spreading and gain-of-stemness. Therefore, eHSP90α and eHSP90α-rich tumor microenvironment can be regarded as novel cancer therapeutic targets. In our previous published results of mouse tumor model, EndoMT-derived CAFs significantly promoted the tumor growth of pancreatic adenocarcinoma cell grafts. However, this EndoMT-promoted tumorigeneity was drastically suppressed when the mice received intravenous injections with mouse anti-HSP90α monoclonal antibody since Day-4 post-cancer cell inoculation. We have sequenced the gene of this mouse anti-HSP90α monoclonal antibody. After computer-assisted analyses and suggestions, we have further constructed a humanized and improved antibody gene. The recombinant gene has been introduced into ExpiCHO cells to express the humanized anti-HSP90α antibody, HH01. Through a series of analyses, we have known that HH01 antibody has a novel amino acid sequence in its CDRs, and exhibits a high binding affinity toward eHSP90α with a KD value of 1.87×10⁻¹⁰ M. HH01 antibody is not easy to form aggregate. It is quite water-soluble but not easy to be excreted out of mouse bodies with a half-life >18.4 days in blood. It is not cytotoxic to retinal pigmented epithelial cells, and will not cause mouse splenic enlargement. HH01 antibody also exhibits its superiority in anti-cancer functions. It potently suppresses invasive and spheroid-forming activities of pancreatic adenocarcinoma and colorectal cancer cell lines. The results of our three mouse cancer models reveal that HH01 antibody alone can abolish EndoMT-promoted desmoplastic tumor-growth activity of subcutaneously inoculated pancreatic adenocarcinoma cell grafts, and can also be used in combination with gemcitabine to exhibit a synergy. In PDAC-developing transgenic mouse model, it can potently suppress K-Ras mutation-caused pancreatic desmoplasia and adenocarcinoma development and liver metastasis, and thus prolong the survival time of experimental mice. Additionally, in M2-macrophage-exacerbated mouse PDAC model, HH01 antibody is also efficacious to suppress the tumor growth and improve the tumor immunity.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

1. An isolated antibody, comprising:
complementarity determining regions (CDRs) capable of specifically binding to HSP90α epitope containing amino acid sequence EDK in amino acid 235 to 244 and amino acid 251 to 260 regions.

2. The isolated antibody of claim 1, wherein the isolated antibody comprises:
heavy chain complementary determining regions CDR1, CDR2 and CDR3 of a heavy chain variable region sequence of SEQ ID NO: 2 or SEQ ID NO: 12; and
light chain complementary determining regions CDR1, CDR2 and CDR3 of a light chain variable region sequence of SEQ ID NO: 7 or SEQ ID NO: 17.

3. The isolated antibody of claim 2, wherein the heavy chain CDR1, CDR2 and CDR3 are from SEQ ID NO: 2, and the light chain CDR1, CDR2 and CDR3 are from SEQ ID NO: 7.

4. The isolated antibody of claim 3, wherein the isolated antibody includes a heavy chain variable region that is at least 80% identical to the sequence of SEQ ID NO: 2, and a light chain variable region that is at least 80% identical to the sequence of SEQ ID NO: 7.

5. The isolated antibody of claim 3, wherein the heavy chain CDR1 has the sequence of SEQ ID NO: 3, the heavy chain CDR2 has the sequence of SEQ ID NO: 4, the heavy chain CDR3 has the sequence of SEQ ID NO: 5, the light chain CDR1 has the sequence of SEQ ID NO: 8, the light chain CDR2 has the sequence of SEQ ID NO: 9, and the light chain CDR3 has the sequence of SEQ ID NO: 10.

6. The isolated antibody of claim 2, wherein the heavy chain CDR1, CDR2 and CDR3 are from SEQ ID NO: 12, and the light chain CDR1, CDR2 and CDR3 are from SEQ ID NO: 17.

7. The isolated antibody of claim 6, wherein the isolated antibody includes a heavy chain variable region that is at least 80% identical to the sequence of SEQ ID NO: 12, and a light chain variable region that is at least 80% identical to the sequence of SEQ ID NO: 17.

8. The isolated antibody of claim 6, wherein the heavy chain CDR1 has the sequence of SEQ ID NO: 13, the heavy chain CDR2 has the sequence of SEQ ID NO: 14, the heavy chain CDR3 has the sequence of SEQ ID NO: 15, the light chain CDR1 has the sequence of SEQ ID NO: 18, the light chain CDR2 has the sequence of SEQ ID NO: 19, and the light chain CDR3 has the sequence of SEQ ID NO: 20.

9. The isolated antibody of claim 1, wherein the isolated antibody is an antibody containing an Fc region, an Fab fragment, an Fab′ fragment, an F(ab′)2 fragment, a single-chain antibody, an scFV multimer, a monoclonal antibody, a monovalent antibody, a multispecific antibody, a humanized antibody, or a chimeric antibody.

10. Nucleic acid molecules containing nucleic acid sequences that encode the isolated antibody of any of claim 1.

11. A pharmaceutical composition, comprising the isolated antibody of claim 1, or the nucleic acid molecules containing nucleic acid sequences that encode said isolated antibody; and a pharmaceutically acceptable carrier.

12. The pharmaceutical composition of claim 11, wherein the isolated antibody comprises the heavy chain CDR1 having the sequence of SEQ ID NO: 13, the heavy chain CDR2 having the sequence of SEQ ID NO: 14, the heavy chain CDR3 having the sequence of SEQ ID NO: 15, the light chain CDR 1 having the sequence of SEQ ID NO: 18, the light chain CDR2 having the sequence of SEQ ID NO: 19, and the light chain CDR3 having the sequence of SEQ ID NO: 20.

13. A method for treating a cancer in a subject, comprising:
administering to the subject an effective amount of the isolated antibody of of claim 1, or the nucleic acid molecules containing nucleic acid sequences that encode said isolated antibody.

14. The method of claim 13, wherein the isolated antibody comprises the heavy chain CDR1 having the sequence of SEQ ID NO: 13, the heavy chain CDR2 having the sequence of SEQ ID NO: 14, the heavy chain CDR3 having the sequence of SEQ ID NO: 15, the light chain CDR1 having the sequence of SEQ ID NO: 18, the light chain CDR2 having the sequence of SEQ ID NO: 19, and the light chain CDR3 having the sequence of SEQ ID NO: 20.

15. The method of claim 13, wherein the cancer has a desmoplasia feature.

16. The method of claim 13, wherein the cancer has a M2-macrophage-exacerbated feature.

17. The method of claim 13, wherein the cancer is pancreatic cancer, colon cancer, breast cancer, liver cancer or lung cancer.

18. The method of claim 13, further comprising administering a therapeutic agent to the subject.

19. The method of claim 18, wherein the therapeutic agent is gemcitabine.

20. The method of claim 13, wherein a blood HSP90α level in the subject is detected using the isolated antibody of claim 1 to monitor the shrink of tumor in the subject.

21. A method for treating desmoplasia in a subject, comprising:
administering to the subject an effective amount of the isolated antibody of claim 1, or the nucleic acid molecules containing nucleic acid sequences that encode said isolated antibody.

22. The method of claim 21, wherein the isolated antibody comprises the heavy chain CDR1 having the sequence of SEQ ID NO: 13, the heavy chain CDR2 having the sequence of SEQ ID NO: 14, the heavy chain CDR3 having the sequence of SEQ ID NO: 15, the light chain CDR1 having the sequence of SEQ ID NO: 18, the light chain CDR2 having the sequence of SEQ ID NO: 19, and the light chain CDR3 having the sequence of SEQ ID NO: 20.

23. The method of claim 21, wherein a blood HSP90□ level in the subject is detected using the isolated antibody of 9 claim 1 to monitor the shrink of tumor in a subject.

24. A method for treating cancer desmoplasia in a subject, comprising:
administering a therapeutic agent targeting eHSP90α to the subject.


SEQUENCE LISTING (20 sequences)

SEQ ID NO: 1 - DNA, 353 bp, Artificial Sequence
  Synthetic polynucleotide
aggtgcaggttgttgagtctggtggaggattggtgcagcctaaagggtcattgaaactctcatgtgcagcctctggattcaccttcaaaacctacgccatgaactgggtccgccaggctccaggaaagggtttggaatgggttgctcgcataagaagtaaaagtagaaattatgcaacatattatgccgattcagtgaaagacaggttcaccatctccagagatgattcacaattcatgttctatctgcaaatgaacaacttgaaaactgaggacacagccatgtattactgtgttgtggggggctcgggttttgactactggggccaaggcaccactctcacagtctcctca

SEQ ID NO: 2 - protein, 118 aa, Artificial Sequence
  Heavy chain variable region
EVQVVESGGGLVQPKGSLKLSCAASGFTFKTYAMNWVRQAPGKGLEWVARIRSKSRNYATYYADSVKDRFTISRDDSQFMFYLQMNNLKTEDTAMYYCVVGGSGFDYWGQGTTLTVSS

SEQ ID NO: 3 - protein, 8 aa, Artificial Sequence
  Heavy chain variable region CDR1
GFTFKTYA

SEQ ID NO: 4 - protein, 10 aa, Artificial Sequence
  Heavy chain variable region CDR2
IRSKSRNYAT

SEQ ID NO: 5 - protein, 9 aa, Artificial Sequence
  Heavy chain variable region CDR3
VVGGSGFDY

SEQ ID NO: 6 - DNA, 336 bp, Artificial Sequence
  Synthetic polynucleotide
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcagcatcagcagagtggaggctggggatctgggagtttattactgctttcaaggttcacatgttccgtggacgttcggtggaggcaccaagctggaaatcaaa

SEQ ID NO: 7 - protein, 107 aa, Artificial Sequence
  Light chain variable region
DVLMTQTPLSLPVSLGDQASISCRSSQSIVHSNGNTYLEWYLQKPGQSPKLLIYKVGVPDRFSGSGSGTDFTLSISRVEAGDLGVYYCFQGSHVPWTFGGGTKLEIK

SEQ ID NO: 8 - protein, 16 aa, Artificial Sequence
  Light chain variable region CDR1
RSSQSIVHSNGNTYLE

SEQ ID NO: 9 - protein, 7 aa, Artificial Sequence
  Light chain variable region CDR2
KVSNRFS

SEQ ID NO: 10 - protein, 9 aa, Artificial Sequence
  Light chain variable region CDR3
FQGSHVPWT

SEQ ID NO: 11 - DNA, 354 bp, Artificial Sequence
  Synthetic polynucleotide
gaggtgcagctggtggagtctggaggaggactggtgcagccaggaggaagcctgagactgtcttgcgccgcttccggcttcacctttaagacatacgccatgaactgggtgaggcaggctcctggacagggactggagtgggtggctaggatccggtctaagtcccggaattacgccacctactatgctgactccgtgaaggatagattcacaatcagccgcgacgattctaagaaccagttttatctgcagatgaacaatctgcgcaccgaggacacagccgtgtactattgcgtggtgggcggcagcggcttcgattattggggccagggcaccacactgaccgtgtccagc

SEQ ID NO: 12 - protein, 118 aa, Artificial Sequence
  Heavy chain variable region
EVQLVESGGGLVQPGGSLRLSCAASGFTFKTYAMNWVRQAPGQGLEWVARIRSKSRNYATYYADSVKDRFTISRDDSKNQFYLQMNNLRTEDTAVYYCVVGGSGFDYWGQGTTLTVSS

SEQ ID NO: 13 - protein, 8 aa, Artificial Sequence
  Heavy chain variable region CDR1
TFKTYAMN

SEQ ID NO: 14 - protein, 19 aa, Artificial Sequence
  Heavy chain variable region CDR2
RIRSKSRNYATYYADSVKD

SEQ ID NO: 15 - protein, 6 aa, Artificial Sequence
  Heavy chain variable region CDR3
GSGFDY

SEQ ID NO: 16 - DNA, 336 bp, Artificial Sequence
  Synthetic polynucleotide
gacgtggtcatgacccagacaccactgtccctgcctgtgagcctgggcgatcaggccagcatctcttgcaggtccagccagtctatcgtgcactccaacggcaatacctacctggagtggtatctgcagaagccaggccagtccccccagctgctgatctacaaggtgagcaacaggttctctggagtgcctgaccggttttccggaagcggatctggcacagatttcaccctgacaatctcttccgtggaggctgaggacctgggcgtgtactattgtttccagggctcccatgtgccatggacctttggcggcggcacaaagctggagatcaag

SEQ ID NO: 17 - protein, 112 aa, Artificial Sequence
  Light chain variable region
DVVMTQTPLSLPVSLGDQASISCRSSQSIVHSNGNTYLEWYLQKPGQSPQLLIYKVSNRFSGVPDRFSGSGSGTDFTLTISSVEAEDLGVYYCFQGSHVPWTFGGGTKLEIK

SEQ ID NO: 18 - protein, 17 aa, Artificial Sequence
  Light chain variable region CDR1
RSSQSIVHSNGNTYYLE

SEQ ID NO: 19 - protein, 7 aa, Artificial Sequence
  Light chain variable region CDR2
KVSNRFS

SEQ ID NO: 20 - protein, 7 aa, Artificial Sequence
  Light chain variable region CDR3
FQGSHVP